(19) United States
(12) Patent Application Publication
(10) Pub. No.: US 2023/0272036 A1
(43) Pub. Date: Aug. 31, 2023

(54) MULTI-SPECIFIC T CELL RECEPTORS

(71) Applicant: OREGON HEALTH & SCIENCE UNIVERSITY, Portland, OR (US)

(72) Inventors: Klaus FRUEH, Portland, OR (US); Louis PICKER, Portland, OR (US); Jonah SACHA, Portland, OR (US); Scott HANSEN, Portland, OR (US); Benjamin BIMBER, Portland, OR (US); Shaheed ABDULHAQQ, Portland, OR (US)

(21) Appl. No.: 17/786,186

(22) PCT Filed: Dec. 15, 2020
(86) PCT No.: PCT/US2020/065147

§ 371 Date: Jun. 16, 2022

Related U.S. Application Data
(60) Provisional application No. 62/948,691, filed on Dec. 16, 2019.

(51) Int. Cl. C07K 14/725; C12N 15/86; C07K 14/74; A61P 35/00

(52) U.S. Cl. CPC C07K 14/7051; C12N 15/86; C07K 14/70539; A61P 35/00; C07K 2317/565

(57) ABSTRACT

The present invention provides CD8+ T cells comprising multi-specific T cell receptors and methods for making the same.

### CROSS REFERENCE TO RELATED APPLICATIONS

This application claims the benefit of U.S. Provisional Application No. 62/948,691, filed Dec. 16, 2019, which is hereby incorporated by reference in its entirety.

### STATEMENT REGARDING FEDERALLY-SPONSORED RESEARCH AND DEVELOPMENT

This invention was made with government support under P01 AI094417, U19 AI128741, RO1 AI117802, and RO1 AI140888 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name: 4153_014PC01_Seqlisting_ST25; Size: 12,548 bytes; and Date of Creation: Nov. 30, 2020) filed with the application is incorporated herein by reference in its entirety.

### BACKGROUND OF THE INVENTION

Conventionally restricted T cell receptors (TCRs) recognize a specific peptide, or epitope, within a given protein, or antigen, that is presented by a specific allele of major histocompatibility complex (MHC) class I or class II. For instance, the mouse T cell receptor OT-1 is specific to the murine MHC-I molecule Kb presenting the peptide SIINFEKL derived from the antigen ovalbumin.

Conventionally restricted TCRs are currently in clinical development for the treatment of cancer and chronic infectious diseases. This is usually achieved by first cloning a TCR specific for a desired antigen that is presented by a common MHC allele. Next, the TCR is introduced into autologous T cells (i.e. T cells derived from a given patient). Upon expansion of these cell in vitro, such “TCR T cells” are re-introduced into the patient for treatment (similar to T cells expressing a chimeric antigen receptor, or CAR). There are several disadvantages to this method: a) autologous TCR T cells have to be generated anew each time for treatment of a new patient, b) the TCR can only be used in humans that express the correct MHC allele, c) since the TCR is highly specific for a given peptide, mutations in the peptide sequence results in escape from TCR recognition. With respect to a) there are numerous efforts in industry and academia to generate “off the shelf” heterologous T cells, i.e., T cell lines that would not be rejected when given to another person. However, there is currently no solution for b) and c).

Occasionally it has been observed that a single TCR can recognize more than one peptide presented by the same or different MHC molecules. However, up to now, no method existed that can specifically generate single TCRs with multiple (unrelated) specificities.

### BRIEF SUMMARY OF THE INVENTION

The present invention relates to a method of generating CD8+ T cells comprising multi-specific TCRs, the method comprising: (a) administering to a subject a recombinant cytomegalovirus (CMV) vector comprising a nucleic acid sequence that encodes a first heterologous antigen, in an amount effective to generate a first set of CD8+ T cells that recognize a first MHC/heterologous antigen-derived peptide complex, wherein the CMV vector does not express an active UL128, UL130, UL146 and UL147 protein or orthologs thereof; (b) identifying a first CD8+ TCR from the first set of CD8+ T cells, wherein the first CD8+ TCR recognizes the first MHC/heterologous antigen-derived peptide complex; (c) administering to the subject a second heterologous antigen in an amount effective to generate a second set of CD8+ T cells that recognizes a second MHC/heterologous antigen-derived peptide complex; (d) isolating one or more CD8+ T cells from the second set of CD8+ T cells; (e) identifying a second CD8+ TCR from the second set of CD8+ T cells, wherein the second CD8+ TCR recognizes the first MHC/heterologous antigen-derived peptide complex and the second MHC/heterologous antigen-derived peptide complex; (f) transfecting a third set of CD8+ T cells with an expression vector, wherein the expression vector comprises a nucleic acid sequence encoding a third CD8+ TCR and a promoter operably linked to the nucleic acid sequence encoding the third CD8+ TCR, wherein the third CD8+ TCR comprises CDR3α and CDR3β of the second CD8+ TCR, thereby generating one or more CD8+ T cells that recognize the first MHC/heterologous antigen-derived peptide complex and the second MHC/heterologous antigen-derived peptide complex; and (g) selecting one or more of the third CD8+ TCRs with the highest avidity for a specific peptide of interest.

In one embodiment, the recombinant CMV vector does not express an active UL18 protein. In one embodiment, the recombinant CMV vector expresses an active UL40 protein, or ortholog thereof, and an active US28 protein, or ortholog thereof.

In one embodiment, the first MHC/heterologous antigen-derived peptide complex is a MHC-II/heterologous antigen-derived peptide complex, a MHC-E/heterologous antigen-derived peptide complex, or a MHC-I/heterologous antigen-derived peptide complex. In one embodiment, the second MHC/heterologous antigen-derived peptide complex is a MHC-II/heterologous antigen-derived peptide complex or a MHC-E/heterologous antigen-derived peptide complex.

In one embodiment, the subject is a human or non-human primate. In one embodiment, the recombinant CMV vector is a recombinant human CMV vector or a recombinant rhesus macaque CMV vector.

In one embodiment, the first and/or second heterologous antigen comprises a tumor antigen, pathogen-specific antigen, a tissue specific antigen, or a host-self antigen. In one embodiment, the tumor antigen is related to a cancer selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer. In one embodiment, the pathogen-specific antigen is related to a pathogen selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, the first CD8+ TCR recognizes specific MHC-II subtopes or supertopes. In one embodiment, the first CD8+ TCR recognizes specific MHC-E subtopes or supertopes. In one embodiment, wherein the first CD8+ TCR recognizes specific MHC-I subtopes or supertopes.

In one embodiment, the first CD8+ TCR is identified by DNA or RNA sequencing. In another embodiment, the first CD8+ TCR is identified by single cell sequencing.

In one embodiment, the first heterologous antigen and second heterologous antigens are the same. In one embodiment, the first heterologous antigen and second heterologous antigen are different.

In one embodiment, the one or more isolated CD8+ T cells from the second set of CD8+ T cells express CD69 and TNFα.

In one embodiment, the second CD8+ TCR recognizes one or more specific supertopes. In one embodiment, the second CD8+ TCR recognizes one or more specific MHC-E supertopes. In one embodiment, the second CD8+ TCR recognizes one or more specific MHC-I supertopes.

In one embodiment, the second CD8+ TCR recognizes a MHC-II supertope and a supertope. In one embodiment, the second CD8+ TCR recognizes a MHC-I supertope and a MHC-E supertope. In one embodiment, the second CD8+ TCR recognizes a MHC-I supertope and a MHC-II supertope.

In one embodiment, the second CD8+ TCR recognizes one or more specific subtopes. In one embodiment, the second CD8+ TCR recognizes one or more specific MHC-E subtopes. In one embodiment, wherein the second CD8+ TCR recognizes one or more specific MHC-I subtopes.

In one embodiment, the second CD8+ TCR recognizes a MHC-II subtope and a subtope. In one embodiment, the second CD8+ TCR recognizes a MHC-II subtope and a MHC-I subtope. In one embodiment, the second CD8+ TCR recognizes a subtope and a MHC-I subtope.

In one embodiment, the second CD8+ TCR recognizes a MHC-II subtope or supertope and a MHC-E subtope or supertope. In one embodiment, the second CD8+ TCR recognizes a MHC-II subtope or supertope and a MHC-I subtope or supertope. In one embodiment, the second CD8+ TCR recognizes a MHC-E subtope or supertope and a subtope or supertope.

In one embodiment, the second CD8+ TCR recognizes specific MHC-II supertopes and MHC-II subtopes. In one embodiment, the second CD8+ TCR recognizes specific supertopes and MHC-E subtopes. In one embodiment, the second CD8+ TCR recognizes specific MHC-I supertopes and MHC-I subtopes.

In one embodiment, the second CD8+ TCR recognizes more than one MHC-II supertope from the same antigen. In one embodiment, the second CD8+ TCR recognizes more than one MHC-E supertope from the same antigen. In one embodiment, the second CD8+ TCR recognizes more than one MHC-I supertope from the same antigen.

In one embodiment, the second CD8+ TCR recognizes more than one MHC-II subtope from the same antigen. In one embodiment, the second CD8+ TCR recognizes more than one MHC-E subtope from the same antigen. In one embodiment, the second CD8+ TCR recognizes more than one MHC-I subtope from the same antigen.

In one embodiment, the second CD8+ TCR recognizes one or more MHC-II supertopes and one or more MHC-II subtopes from the same antigen. In one embodiment, the second CD8+ TCR recognizes one or more MHC-E supertopes and one or more MHC-E subtopes from the same antigen. In one embodiment, the second CD8+ TCR recognizes one or more MHC-I supertopes and one or more MHC-I subtopes from the same antigen.

In one embodiment, the second CD8+ TCR recognizes more than one MHC-II supertope from more than one antigen. In one embodiment, the second CD8+ TCR recognizes more than one MHC-E supertope from more than one antigen. In one embodiment, the second CD8+ TCR recognizes more than one MHC-I supertope from more than one antigen.

In one embodiment, the second CD8+ TCR recognizes more than one MHC-II subtope from more than one antigen. In one embodiment, the second CD8+ TCR recognizes more than one MHC-E subtope from more than one antigen. In one embodiment, the second CD8+ TCR recognizes more than one MHC-I subtope from more than one antigen.

In one embodiment, the second CD8+ TCR recognizes one or more MHC-II supertopes and one or more MHC-II subtopes from different antigens. In one embodiment, the second CD8+ TCR recognizes one or more MHC-E supertopes and one or more MHC-E subtopes from different antigens. In one embodiment, the second CD8+TCR recognizes one or more MHC-I supertopes and one or more MHC-I subtopes from different antigens.

In one embodiment, the third CD8+ TCR recognizes one or more specific MHC-II supertopes. In one embodiment, the third CD8+ TCR recognizes one or more specific MHC-E supertopes. In one embodiment, the third CD8+ TCR recognizes one or more specific MHC-I supertopes.

In one embodiment, the third CD8+ TCR recognizes one or more specific MHC-II subtopes. In one embodiment, the third CD8+ TCR recognizes one or more specific MHC-E subtopes. In one embodiment, the third CD8+ TCR recognizes one or more specific MHC-I subtopes.

In one embodiment, the third CD8+ TCR recognizes specific MHC-II supertopes and MHC-II subtopes. In one embodiment, the third CD8+ TCR recognizes specific MHC-E supertopes and MHC-E subtopes. In one embodiment, the third CD8+ TCR recognizes specific MHC-I supertopes and MHC-I subtopes.

In one embodiment, the third CD8+ TCR recognizes more than one MHC-II supertope from one antigen. In one embodiment, the third CD8+ TCR recognizes more than one MHC-E supertope from one antigen. In one embodiment, the third CD8+ TCR recognizes more than one MHC-I supertope from one antigen.

In one embodiment, the third CD8+ TCR recognizes more than one MHC-II subtope from one antigen. In one embodiment, the third CD8+ TCR recognizes more than one MHC-E subtope from one antigen. In one embodiment, wherein the third CD8+ TCR recognizes more than one MHC-I subtope from one antigen.

In one embodiment, the third CD8+ TCR recognizes one or more MHC-II supertopes and one or more MHC-II subtopes from one antigen. In one embodiment, the third CD8+ TCR recognizes one or more MHC-E supertopes and one or more MHC-E subtopes from one antigen. In one embodiment, the third CD8+ TCR recognizes one or more MHC-I supertopes and one or more MHC-I subtopes from one antigen.

In one embodiment, the third CD8+ TCR recognizes more than one MHC-II supertope from more than one antigen. In one embodiment, the third CD8+ TCR recognizes more than one MHC-E supertope from more than one antigen. In one embodiment, the third CD8+ TCR recognizes more than one MHC-E supertope from more than one antigen.

In one embodiment, the third CD8+ TCR recognizes more than one MHC-II subtope from more than one antigen. In one embodiment, the third CD8+ TCR recognizes more than one MHC-E subtope from more than one antigen. In one embodiment, the third CD8+ TCR recognizes more than one MHC-I subtope from more than one antigen.

In one embodiment, the third CD8+ TCR recognizes specific MHC-E subtopes or supertopes and MHC-II subtopes or supertopes. In one embodiment, the third CD8+ TCR recognizes specific MHC-E subtopes or supertopes and MHC-I subtopes or supertopes. In one embodiment, the third CD8+ TCR recognizes specific MHC-II subtopes or supertopes and MHC-I subtopes or supertopes.

In one embodiment, the third CD8+ TCR recognizes more than one MHC-II subtope from the same antigen. In one embodiment, the third CD8+ TCR recognizes more than one MHC-E subtope from the same antigen. In one embodiment, the third CD8+ TCR recognizes more than one MHC-I subtope from the same antigen.

In one embodiment, the third CD8+ TCR recognizes one or more MHC-II supertopes and one or more MHC-II subtopes from different antigens. In one embodiment, the third CD8+ TCR recognizes one or more MHC-E supertopes and one or more MHC-E subtopes from different antigens. In one embodiment, the third CD8+ TCR recognizes one or more MHC-I supertopes and one or more MHC-I subtopes from different antigens.

In one embodiment, the nucleic acid sequence encoding the third CD8+ TCR is identical to the nucleic acid sequence encoding the second CD8+ TCR.

In one embodiment, one or more CD8+ T cells are isolated from a second subject and transfecting the one or more CD8+ T cells with a nucleic acid sequence encoding the selected third CD8+ TCR and a promoter operably linked to the nucleic acid sequence encoding the third CD8+ TCR, thereby generating one or more CD8+ T cells that recognize the first MHC/heterologous antigen-derived peptide complex and the second MHC/heterologous antigen-derived peptide complex.

In one embodiment, the first MHC-heterologous antigen-derived peptide complex is a MHC-II/heterologous antigen-derived peptide complex, a MHC-E/heterologous antigen-derived peptide complex, or a MHC-I/heterologous antigen-derived peptide complex. In one embodiment, the second MHC-heterologous antigen-derived peptide complex is a MHC-II/heterologous antigen-derived peptide complex, a MHC-E/heterologous antigen-derived peptide complex, or a MHC-I/heterologous antigen-derived peptide complex.

In one embodiment, the transfected CD8+ T cells are administered to the second subject to treat or prevent cancer. In another embodiment, the cancer is selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer.

In one embodiment, the transfected CD8+ T cells are administered to the second subject to treat a pathogenic-infection

In another embodiment, the pathogenic infection is selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, the first subject is a nonhuman primate and the second subject is a human, and wherein the transfected CD8+ T cells comprises a chimeric nonhuman primate-human CD8+ TCR comprising the non-human primate CDR3α and CDR3β of the second CD8+ TCR.

In one embodiment, the third CD8+ TCR comprises the non-human primate CDR1α, CDR2α, CDR3α, CDR1β, CDR2β, and CDR3β of the second CD8+ TCR. In one embodiment, the third CD8+ TCR comprises the CDR1α, CDR2α, CDR3α, CDR1β, CDR2β, and CDR3β of the second CD8+ TCR.

In one embodiment, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR.

In one embodiment, the third CD8+ TCR is a chimeric CD8+ TCR.

In one embodiment, administering the recombinant CMV vector to the first subject comprises intravenous, intramuscular, intraperitoneal, or oral administration.

In one embodiment, a CD8+ T cell comprising the multi-specific TCR is generated by the method.

In one embodiment, the CD8+ T cell is administered to a subject in need thereof to treat or prevent cancer. In another embodiment, the cancer is selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer.

In one embodiment, the CD8+ T cell is administered to a subject in need thereof to treat a pathogenic infection. In another embodiment, the pathogenic infection is selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, the recombinant CMV vector to the first subject comprises intravenous, intramuscular, intraperitoneal, or oral administration.

The present invention also relates to a method of generating CD8+ T cells comprising a multi-specific T cell receptor (TCR) comprising: (a) administering to a subject a recombinant cytomegalovirus (CMV) vector comprising a nucleic acid sequence that encodes a first heterologous antigen, in an amount effective to generate a first set of CD8+ T cells that recognize a first MHC-E/heterologous antigen-derived peptide complex, wherein the CMV vector does not express an active UL128, UL130, UL146 and UL147 protein or orthologs thereof, and wherein the recombinant CMV vector further comprises a microRNA recognition element (MRE); (b) identifying a first CD8+ TCR from the first set of CD8+ T cells, wherein the first CD8+ TCR recognizes the first MHC-E/heterologous antigen-derived peptide complex; (c) administering to the subject a second heterologous antigen in an amount effective to generate a second set of CD8+ T cells that recognizes a second MHC-E/heterologous antigen-derived peptide complex; (d) isolating one or more CD8+ T cells from the second set of CD8+ T cells; (e) identifying a second CD8+ TCR from the second set of CD8+ T cells, wherein the second CD8+ TCR recognizes the first MHC-E/heterologous antigen-derived peptide complex and the second MHC-E/heterologous antigen-derived peptide complex; (f) transfecting a third set of CD8+ T cells with an expression vector, wherein the expression vector comprises a nucleic acid sequence encoding a third CD8+ TCR and a promoter operably linked to the nucleic acid sequence encoding the third CD8+ TCR, wherein the third CD8+ TCR comprises CDR3α and CDR3β of the second CD8+ TCR, thereby generating one or more CD8+ T cells that recognize the first MHC-E/heterologous antigen-derived peptide complex and the second MHC-E/heterologous antigen-derived peptide complex; and (g) selecting one or more of the third CD8+ TCRs with the highest avidity for the specific peptide of interest.

In one embodiment, the subject is a human or non-human primate. In one embodiment, the recombinant CMV vector is a recombinant human CMV vector or a recombinant rhesus macaque CMV vector.

In one embodiment, the first heterologous antigen comprises a tumor antigen, pathogen-specific antigen, a tissue specific antigen, or a host-self antigen. In one embodiment, the tumor antigen is related to a cancer selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer. In one embodiment, the pathogen-specific antigen is related to a pathogen selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, the MRE contains target sites for microRNAs expressed in endothelial cells. In another embodiment, the MRE is specific for the miRNA selected from the group consisting of miR126, miR-126-3p, miR-130a, miR-210, miR-221/222, miR-378, miR-296, and miR-328.

In one embodiment, the first CD8+ TCR recognizes specific MHC-E subtopes or supertopes.

In one embodiment, the first CD8+ TCR is identified by DNA or RNA sequencing. In one embodiment, the first CD8+ TCR is identified by single cell sequencing.

In one embodiment, the second heterologous antigen comprises a tumor antigen, pathogen-specific antigen, a tissue specific antigen, or a host-self antigen. In one embodiment, the tumor antigen is related to a cancer selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer. In one embodiment, the pathogen-specific antigen is related to a pathogen selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, the first heterologous antigen and second heterologous antigens are the same. In one embodiment, the first heterologous antigen and second heterologous antigen are different.

In one embodiment, the one or more isolated CD8+ T cells from the second set of CD8+ T cells express CD69 and TNFα.

In one embodiment, the second CD8+ TCR is identified by DNA or RNA sequencing. In one embodiment, the second CD8+ TCR is identified by single cell sequencing.

In one embodiment, the second CD8+ TCR recognizes one or more specific MHC-E supertopes. In one embodiment, the second CD8+ TCR recognizes one or more specific MHC-E subtopes. In one embodiment, the second CD8+ TCR recognizes specific MHC-E supertopes and MHC-E subtopes.

In one embodiment, the second CD8+ TCR recognizes more than one MHC-E supertope from the same antigen. In one embodiment, the second CD8+ TCR recognizes more than one MHC-E subtope from the same antigen.

In one embodiment, the second CD8+ TCR recognizes one or more MHC-E supertopes and one or more MHC-E subtopes from the same antigen. In one embodiment, the second CD8+ TCR recognizes more than one MHC-E supertope from more than one antigen. In one embodiment, the second CD8+ TCR recognizes more than one MHC-E subtope from more than one antigen. In one embodiment, the second CD8+ TCR recognizes one or more MHC-E supertopes and one or more MHC-E subtopes from different antigens.

In one embodiment, the third CD8+ TCR recognizes one or more specific MHC-E supertopes. In one embodiment, the third CD8+ TCR recognizes one or more specific MHC-E subtopes. In one embodiment, the third CD8+ TCR recognizes specific MHC-E supertopes and MHC-E subtopes.

In one embodiment, the third CD8+ TCR recognizes more than one MHC-E supertope from one antigen. In one embodiment, the third CD8+ TCR recognizes more than one MHC-E subtope from one antigen.

In one embodiment, the third CD8+ TCR recognizes one or more MHC-E supertopes and one or more MHC-E subtopes from one antigen. In one embodiment, the third CD8+ TCR recognizes more than one MHC-E supertope from more than one antigen.

In one embodiment, the third CD8+ TCR recognizes more than one MHC-E subtope from more than one antigen. In one embodiment, the third CD8+ TCR recognizes specific MHC-E supertopes and MHC-E subtopes.

In one embodiment, the third CD8+ TCR recognizes more than one MHC-E subtope from the same antigen. In one embodiment, the third CD8+ TCR recognizes one or more MHC-E supertopes and one or more MHC-E subtopes from different antigens.

In one embodiment, the nucleic acid sequence encoding the third CD8+ TCR is identical to the nucleic acid sequence encoding the second CD8+ TCR.

In one embodiment, one or more CD8+ T cells are isolated from a second subject and transfecting the one or more CD8+ T cells with a nucleic acid sequence encoding the selected third CD8+ TCR and a promoter operably linked to the nucleic acid sequence encoding the third CD8+ TCR, thereby generating one or more CD8+ T cells that recognize the first MHC-E/heterologous antigen-derived peptide complex and the second MHC-E/heterologous antigen-derived peptide complex.

In one embodiment, the transfected CD8+ T cells are administered to the second subject to treat or prevent cancer. In another embodiment, the cancer is selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer.

In one embodiment, the transfected CD8+ T cells are administered to the second subject to treat a pathogenic-infection. In another embodiment, the pathogenic infection is selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, the first subject is a nonhuman primate and the second subject is a human, and wherein the transfected CD8+ T cells comprises a chimeric nonhuman primate-human CD8+ TCR comprising the non-human primate CDR3α and CDR3β of the second CD8+ TCR.

In one embodiment, the third CD8+ TCR comprises the non-human primate CDR1α, CDR2α, CDR3α, CDR1β, CDR2β, and CDR3β of the second CD8+ TCR. In one embodiment, the third CD8+ TCR comprises the CDR1α, CDR2α, CDR3α, CDR1β, CDR2β, and CDR3β of the second CD8+ TCR.

In one embodiment, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR.

In one embodiment, the third CD8+ TCR is a chimeric CD8+ TCR.

In one embodiment, administering the recombinant CMV vector to the first subject comprises intravenous, intramuscular, intraperitoneal, or oral administration.

In one embodiment, a CD8+ T cell comprising the multi-specific TCR is generated by the method.

In one embodiment, the CD8+ T cell is administered to a subject in need thereof to treat or prevent cancer. In one embodiment, the cancer is selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer.

In one embodiment, the CD8+ T cell is administered to a subject in need thereof to treat a pathogenic infection. In one embodiment, the pathogenic infection is selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In one embodiment, administering the recombinant CMV vector to the first subject comprises intravenous, intramuscular, intraperitoneal, or oral administration.

### BRIEF DESCRIPTION OF THE DRAWINGS/FIGURES

FIGS. 1A-B show an overview of the primary study cohort. FIG. 1A is a timeline showing the vaccination dates and sampling window used in this study. FIG. 1B shows the overlapping 15 SIVgag peptides recognized by rhesus macaques (RM) using intracellular cytokine staining (ICS) with each T cell-targeted peptide “box” colored based on their MHC restriction as determined by differentially blocking analysis. Green=MHC-E, red=MHC-la, blue=MHC-II, purple=indeterminate. The MHC-E and MHC-II restricted supertopes are labeled.

FIG. 2A-F show TCR clonotypic hierarchies of MHC-E supertope responses.

FIG. 2A shows peripheral blood mononuclear cells (PBMC) from RhCMV 68-1/SIVgag-vaccinated RM (Rh-1) that were stimulated with EK9 peptide in the presence of the secretion inhibitor Brefeldin A and intracellular cytokine (TNF-α v. IFN-γ) analysis (ICS) was performed to identify EK9-specific CD8+ T cells (left). In parallel, the same PBMC were stimulated with EK9 in the absence of Brefeldin A, but in the presence of TAPI-0, an inhibitor of TNF-α cleavage, with responding cells identified by activation-induced upregulation of surface CD69 and surface-trapped TNF-α (STTS analysis; right). FIG. 2B are bar plots illustrating the clonotypic hierarchies for each time point, based on CDR3 alpha and/or beta sequence. In many cases, a given TCR α/β pair clone was found in the responsive fraction after both EK9 and RL9 stimulation (asterisks). FIG. 2F shows a representative ICS experiment, in which these transductants were cultured with BLCL pulsed with no peptide, a negative control peptide, Gag RL9, or Gag EK9.

FIGS. 3A-3B show the SIVgag recognition by TCR transductants. FIG. 3A are the results of a flow cytometry experiment showing target cells that were generated by infecting purified SEB/CD3-activated Rh-4 CD4+ T cells with SIVmac239 or transducing Rh-5 (Mamu-A*01+) BLCL with a retrovirus the expressing both SIV Gag and truncated NGFR, which provides a surface marker (NGFR-T2A-Gag). FIG. 3B are ICS assays with the target cells and the indicated MHC-E-TCR CD8+ T cell transductants. CD8+ T cell transductants expressing a Mamu-A*01-restricted, CM9-specific TCR were used as a positive control. Non-transduced CD8+ T cells or CD8+ T cell transductants expressing an (irrelevant) MR1-restricted TCR were used as negative controls.

FIGS. 4A-4D show pie charts demonstrating the complete clonotypic hierarchies for SIV-infected recognition.

FIG. 5 shows the analysis of epitope cross-reactivity using TCR transductants. Representative ICS using CD8+ T cell transductants expressing Rh-1 MHC-E-TCR4 vs. TCR 6-1. These transductants were cultured with RM BLCL pulsed with SIVgag MHC-E optimal supertope and subtope peptides for Rh-1, as indicated. Responses were measured using IFN-γ and TNF-α staining.

FIGS. 6A-6B show the response of CD8+ T cells expressing TCRs with MHC-E-presented SIVgag peptide recognition to MHC-II-presented SIVgag peptides and with peptides from an unrelated TB antigen. PBMC from Rh-4 was stimulated with either of the MHC-II supertope peptides Gag₂₁₁₋₂₂₂ (53) or Gag₂₉₀₋₃₀₁ (73) (FIG. 6A) or with a pool of overlapping 15mer peptides from the TB protein Ag85B (FIG. 6B). Activated cells were sorted based on sCD69 and stTNF-α, and TCRs were characterized by scRNAseq.

FIGS. 7A-7J show the cross-reaction of MHC-E-restricted TCRs with CMV IE peptides presented by MHC-Ia. FIG. 7A is a flow cytometry experiment to analyze the response of the four RM to AN10 and VY9 tetramers. FIG. 7B-7E are graphs showing the clonotypic hierarchies for each peptide-specific response identified by both approaches in each RM (note concordance of TCR identification by both approaches). PBMC from each RM was either pulsed with the indicated CMV peptide (AN10 or VY9) followed by STTS, or stained with AN10 or VY9 tetramers, with the reactive CD8+ T cells sorted and analyzed by scRNAseq. FIG. 7F shows ICS analysis of CD8+ transductants expressing TCR2 (top) or TCR4 (bottom) cultured with (Mamu-A*02+ and MHC-E+) BLCL pulsed with the indicated peptide. FIGS. 7G-7J are pie charts showing the clonotypic hierarchies from SIV-infected cell recognition assays, identical to FIG. 4, except TCR clones are shaded based on whether they cross-react with AN10/VY9 or not.

FIGS. 8A-8B show validation of MHC-Ia restriction by VY9 blocking. FIG. 8A shows CD8+ transductants expressing TCR were cultured with Mamu-A*02+ and MHC-E+ BLCL pulsed with the indicated peptide (top row). In parallel, the BLCL were pre-incubated with the strongly MHC-E-binding VL9 peptide prior to pulsing with the epitopic peptide to assess MHC-E restriction of the individual responses (bottom row). FIG. 8b shows and ICS assay from BLCL were pre-incubated with peptides with varying affinity for Mamu-A*02 (CM9=non-A*02 binder; GY9=weak A*02 binder; YY9=strong A*02 binder). After pre-treated cells were pulsed with the AN10 peptide, these BLCL were then used as APCs for the ICS assay using CD8+ T cell transductions expressing TCR14.

FIG. 9 shows ICS demonstrating the specificity analysis of dual-TCR expressing clonotypes.

FIGS. 10A-10B show the functional avidity analysis of MHC-Ia- and MHC-E-restricted responses mediated by the same TCR. Mamu-A1*002 BLCL were pulsed with ten-fold dilutions EK9 or VY9 peptides starting at 200 μM. BLCL were washed and incubated with TCR2 CD8+ T cell transductants in three separate experiments. FIG. 10A shows representative flow cytometric data from one experiment. FIG. 10B is a graph showing the results from all the experiments.

FIGS. 11A-11G shows the transcriptomic response of MHC-E-restricted, SIVgag-reactive CD8+ T cells, with and without MHC-Ia-IE epitope cross-reactivity. FIG. 11A shows a tSNE plot of scRNA-seq of purified CD8+ T cells incubated with BLCL pulsed with EK9 and RL9. Cells were clustered based on transcriptional profile. Colors denote the results of unsupervised clustering. Dots indicate cells expressing previously identified TCR pairs previously identified as MHC-E-restricted (FIG. 4). Cells bearing these MHC-E/SIVgag-specific clones are strikingly enriched in the same cluster (designated by red box). FIG. 11B is a heatmap of the scRNA-seq data. FIG. 11C shows the activation score in the tSNE plot. The activation score was calculated based on the combined expression of nine canonical marker genes [IFNG, MIP-1B (CCL4), TNFRSF9, NFKBID, IRF8, CD83, CD82, PLEK, and RGCC]. FIG. 11D shows the gating of total CD69+ cells. FIG. 11E shows the tSNE plot of scRNA-seq of purified CD8+ T cells incubated with BLCL pulsed with EK9 and RL9. FIG. 11F shows the activation score in the tSNE plot. FIG. 11G are graphs showing the activation score of the CD8+ T cells expressing each indicated TCR to each indicated antigen stimulus. Dotted blue line denotes the threshold at which cells are considered activated.

### DETAILED DESCRIPTION OF THE INVENTION

### I. Terms

Unless otherwise noted, technical terms are used according to conventional usage.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

Although methods and materials similar or equivalent to those described herein may be used in the practice or testing of this disclosure, suitable methods and materials are described below. In addition, the materials, methods, and examples are illustrative only and not intended to be limiting. In order to facilitate review of the various embodiments of the disclosure, the following explanations of specific terms are provided.

Antigen: As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

Antigen-specific T cell: A CD8⁺ or CD4⁺ lymphocyte that recognizes a particular antigen. Generally, antigen-specific T cells specifically bind to a particular antigen presented by MHC molecules, but not other antigens presented by the same MHC.

Administration: As used herein, the term “administration” means to provide or give a subject an agent, such as a composition comprising an effective amount of a CMV vector comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Avidity: As used herein, the term “avidity” refers to the strength of multiple affinities of individual non-covalent binding interactions such as antigen-antibody interactions. Avidity therefore gives a measure for the overall strength of an antigen-antibody complex.

Effective amount: As used herein, the term “effective amount” refers to an amount of an agent, such as a CMV vector comprising a heterologous antigen or a transfected CD8+ T cell that recognizes a MHC-E/heterologous antigen-derived peptide complex, a MHC-II/heterologous antigen-derived peptide complex, or a MHC-I/heterologous antigen-derived peptide complex, that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

Epitope: As used herein, the term “epitope” refers to molecular structure which may completely make up a specific binding partner or be part of a specific binding partner to the binding domain or the T-cell receptor domain polypeptide of the present invention. Chemically, an epitope may either be composed of a carbohydrate, a peptide, a fatty acid, an organic, biochemical or inorganic substance or derivatives thereof and any combinations thereof. If an epitope is a polypeptide, it will usually include at least 3 amino acids, preferably 8 to 50 amino acids, and more preferably between about 10-20 amino acids in the peptide. There is no critical upper limit to the length of the peptide, which could comprise nearly the full length of a polypeptide sequence. Epitopes can be either linear or conformational epitopes. A linear epitope is comprised of a single segment of a primary sequence of a polypeptide chain. Linear epitopes can be contiguous or overlapping. Conformational epitopes are comprised of amino acids brought together by folding of the polypeptide to form a tertiary structure and the amino acids are not necessarily adjacent to one another in the linear sequence. Specifically, epitopes are at least part of diagnostically relevant molecules, i.e. the absence or presence of an epitope in a sample is qualitatively or quantitatively correlated to either a disease or to the health status of a patient or to a process status in manufacturing or to environmental and food status. Epitopes may also be at least part of therapeutically relevant molecules, i.e. molecules which can be targeted by the specific binding domain which changes the course of the disease.

Heterologous antigen: As used herein, the term “heterologous antigen” refers to any protein or fragment thereof that is not derived from CMV. Heterologous antigens may be pathogen-specific antigens, tumor virus antigens, tumor antigens, host self-antigens, or any other antigen.

Immunogenic peptide: A peptide which comprises an allele-specific motif or other sequence, such as an N-terminal repeat, such that the peptide will bind an MHC molecule and induce a cytotoxic T lymphocyte (“CTL”) response, or a B cell response (for example antibody production) against the antigen from which the immunogenic peptide is derived.

In one embodiment, immunogenic peptides are identified using sequence motifs or other methods, such as neural net or polynomial determinations known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In one embodiment, a conserved residue is one where the WIC structure may provide a contact point with the immunogenic peptide.

Mutation: As used herein, the term “mutation” refers to any difference in a nucleic acid or polypeptide sequence from a normal, consensus, or “wild type” sequence. A mutant is any protein or nucleic acid sequence comprising a mutation. In addition, a cell or an organism with a mutation may also be referred to as a mutant. Some types of coding sequence mutations include point mutations (differences in individual nucleotides or amino acids); silent mutations (differences in nucleotides that do not result in an amino acid changes); deletions (differences in which one or more nucleotides or amino acids are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence.

Nucleotide sequences or nucleic acid sequences: The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex.

Operably Linked: As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

Promoter: As used herein, the term “promoter” may refer to any of a number of nucleic acid control sequences that directs transcription of a nucleic acid. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector.

Recombinant: As used herein, the term “recombinant” with reference to a nucleic acid or polypeptide refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

Pharmaceutically acceptable carriers: As used herein, a “pharmaceutically acceptable carrier” of use is conventional. Remington's Pharmaceutical Sciences, by E. W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Polynucleotide: As used herein, the term “polynucleotide” refers to a polymer of ribonucleic acid (RNA) or deoxyribonucleic acid (DNA). A polynucleotide is made up of four bases; adenine, cytosine, guanine, and thymine/uracil (uracil is used in RNA). A coding sequence from a nucleic acid is indicative of the sequence of the protein encoded by the nucleic acid.

Polypeptide: The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

Orthologs of proteins are typically characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

Sequence identity/similarity: As used herein, the identity/similarity between two or more nucleic acid sequences, or two or more amino acid sequences, is expressed in terms of the identity or similarity between the sequences. Sequence identity may be measured in terms of percentage identity; the higher the percentage, the more identical the sequences are. Sequence similarity may be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) may be called “homologs.”

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer App Biosci 8, 155-165 (1992); and Pearson et al, Meth Mol Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) (Altschul et al, (1990) supra) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information may be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment may be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and may possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence may be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

Specifically Binds: As used herein, the term “specifically binds” or “specific binding” refers to a binding reaction which is determinative of the cognate ligand of interest in a heterogeneous population of molecules. Thus, under designated conditions (e.g. immunoassay conditions), the specified T-cell receptor domain polypeptide binds to its particular “target” and does, not bind in a significant amount to other molecules present in a sample.

Subject: As used herein, the term “subject” refers to a living multi-cellular vertebrate organisms, a category that includes both human and non-human mammals.

Subtope: As used herein, the term “subtope” refers to a subdominant epitope or peptide that is recognized by T cells.

Supertope: As used herein, the term “supertope” or “supertope peptide” refers to a epitope or peptide that is recognized by T cells in greater than about 90% of the population regardless of MHC haplotype, i.e., in the presence or absence of given MHC-I, MHC-II, or MHC-E alleles.

Treatment: As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

Vaccine: An immunogenic composition that can be administered to a mammal, such as a human, to confer immunity, such as active immunity, to a disease or other pathological condition. Vaccines can be used prophylactically or therapeutically. Thus, vaccines can be used reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In particular embodiments, a vaccine is a replication-deficient CMV expressing a heterologous antigen, such as a tumor associated antigen derived from a tumor of the lung, prostate, ovary, breast, colon, cervix, liver, kidney, bone, or a melanoma.

Vector: Nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus.

T-Cell Receptor: As used herein, the term “T-Cell receptor” refers to a heterodimeric molecule comprising an alpha polypeptide chain (alpha chain) and a beta polypeptide chain (beta chain), wherein the heterodimeric receptor is capable of binding to a peptide antigen presented by an HLA molecule.

Multiple-Specific T-Cell Receptor: As used herein, the term “multiple-specific T-cell receptor” refers to a T-cell receptor that is capable of binding to multiple peptide antigens. The peptide antigens may be from the same or different antigens. The peptide antigens may be presented by the same or different HLA molecules.

### II. Multi-Specific T Cell Receptors (TCRs)

The present invention is directed to TCRs with multiple specificities to unrelated peptides. T cells bearing these TCRs can be used in patient treatments.

The present invention is also directed to a method of generating CD8+ T cells comprising a multi-specific T cell receptor (TCR), wherein the method comprises administering to a subject a recombinant CMV vector comprising a nucleic acid sequence that encodes a first heterologous antigen, in an amount effective to generate a first set of CD8+ T cells that recognize a first MHC/heterologous antigen-derived peptide complex, wherein the CMV vector does not express an active UL128, UL130, UL146 and UL147 protein or orthologs thereof; identifying a first CD8+ TCR from the first set of CD8+ T cells, wherein the first CD8+ TCR recognizes the first MHC/heterologous antigen-derived peptide complex; administering to the subject a second heterologous antigen in an amount effective to generate a second set of CD8+ T cells that recognizes a second MHC/heterologous antigen-derived peptide complex; isolating one or more CD8+ T cells from the second set of CD8+ T cells; identifying a second CD8+ TCR from the second set of CD8+ T cells, wherein the second CD8+ TCR recognizes the first MHC/heterologous antigen-derived peptide complex and the second MHC/heterologous antigen-derived peptide complex; transfecting a third set of CD8+ T cells with an expression vector, wherein the expression vector comprises a nucleic acid sequence encoding a third CD8+ TCR and a promoter operably linked to the nucleic acid sequence encoding the third CD8+ TCR, wherein the third CD8+ TCR comprises CDR3α and CDR3β of the second CD8+ TCR, thereby generating one or more CD8+ T cells that recognize the first MHC/heterologous antigen-derived peptide complex and the second MHC/heterologous antigen-derived peptide complex; and selecting one or more of the third CD8+ TCRs with the highest avidity for a specific peptide of interest.

Rhesus Cytomegalovirus (RhCMV) vectors lacking functional expression of the RhCMV homologues of human CMV UL128, UL130, UL146 and UL147 while expressing the homologs of UL40 and US28 efficiently elicit broadly targeted Mamu E-restricted CD8+ T cell responses in rhesus monkeys to virtually any protein expressed by this vector, including both RhCMV proteins and exogenous protein inserts, the latter including bacterial, viral and self-protein.

In some embodiments, the subject is a human or non-human primate. In some embodiments, the recombinant CMV vector is a recombinant human CMV vector or a recombinant rhesus macaque CMV vector. In some embodiments, the recombinant CMV does not express an active UL128, UL130, UL146 and UL147 protein due to the presence of a mutation in the nucleic acid sequence encoding UL128, UL130, UL146 and UL147 or homologs thereof, or orthologs thereof (homologous genes of CMV that infect other species). In some embodiments, the recombinant CMV does not express an active UL128, UL130, UL146, UL147, and UL18 protein due to the presence of a mutation in the nucleic acid sequence encoding UL128, UL130, UL146, UL147, and UL18 or homologs thereof, or orthologs thereof (homologous genes of CMV that infect other species). The mutation may be any mutation that results in a lack of expression of the active UL128, UL130, UL146, UL147 or US18 proteins. Such mutations may include point mutations, frameshift mutations, deletions of less than all of the sequence that encodes the protein (truncation mutations), or deletions of all of the nucleic acid sequence that encodes the protein, or any other mutations. Exemplary vectors are described in U.S. Pat. Nos. 9,783,823 and 9,862,972, and US Appl. Pub. No. 2018/0298404 which are herein incorporated by reference.

In some embodiments, the recombinant CMV vector does not express an active UL128, UL130, UL146 and UL147 protein, or homologs thereof, or orthologs thereof, and expresses an active UL40 and US28 protein, or homologs thereof, or orthologs thereof. In some embodiments, the recombinant CMV vector does not express an active UL128, UL130, UL146, UL147, and UL18 protein, or homologs thereof, or orthologs thereof, and expresses an active UL40 and US28 protein, or homologs thereof, or orthologs thereof.

In some embodiments, the first MHC/heterologous antigen-derived peptide complex is a MHC-II/heterologous antigen-derived peptide complex, a MHC-E/heterologous antigen-derived peptide complex, or a MHC-I/heterologous antigen-derived peptide complex. In some embodiments, the second MHC/heterologous antigen-derived peptide complex is a MHC-II/heterologous antigen-derived peptide complex, a MHC-E/heterologous antigen-derived peptide complex, or a MHC-I/heterologous antigen-derived peptide complex.

Human or animal CMV vectors, when used as expression vectors, are innately non-pathogenic in the selected subjects such as humans. In some embodiments, the CMV vectors have been modified to render them non-pathogenic (incapable of host-to-host spread) in the selected subjects.

A heterologous antigen can be any protein or fragment thereof that is not derived from CMV, including tumor antigens, pathogen-specific antigens, model antigens (such as lysozyme, keyhole-limpet hemocyanin (KLH), or ovalbumin), tissue-specific antigens, host self-antigens, or any other antigen.

Pathogen specific antigens can be derived from any human or animal pathogen. The pathogen may be a viral pathogen and the antigen may be a protein derived from the viral pathogen. Viruses include, but are not limited to retroviruses, polyomaviruses, Adenovirus, coxsackievirus, hepatitis A virus, poliovirus, rhinovirus, Herpes simplex, type 1, Herpes simplex, type 2, Varicella-zoster virus, Epstein-Barr virus, Kaposi's sarcoma herpesvirus, Human cytomegalovirus, Human herpesvirus, type 8, Hepatitis B virus, Hepatitis C virus, yellow fever virus, dengue virus, West Nile virus, Human immunodeficiency virus (HIV), Influenza virus, Measles virus, Mumps virus, Parainfluenza virus, Respiratory syncytial virus, Human metapneumovirus, Human papillomavirus, Rabies virus, Rubella virus, Human bocavirus, human T-lymphotropic virus (HTLV1), merkel cell polyomavirus (MCV), cytomegalovirus, and Parvovirus B19.

The pathogen may be a bacterial pathogen and the antigen may be a protein derived from the bacterial pathogen. The pathogenic bacteria include, but are not limited to, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtherias, Enterococcus faecalis, Enterococcus faecium, Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholera and Yersinia pestis.

The pathogen may be a parasite and the antigen may be a protein derived from the parasite pathogen. The parasite may be a protozoan organism or a protozoan organism causing a disease such as, but not limited to, Acanthamoeba, Babesiosis, Balantidiasis, Blastocystosis, Coccidia, Dientamoebiasis, Amoebiasis, Giardia, Isosporiasis, Leishmaniasis, Primary amoebic meningoencephalitis (PAM), Malaria, Rhinosporidiosis, Toxoplasmosis—Parasitic pneumonia, Trichomoniasis, Sleeping sickness and Chagas disease. The parasite may be a helminth organism or worm or a disease caused by a helminth organism such as, but not limited to, Ancylostomiasis/Hookworm, Anisakiasis, Roundworm-Parasitic pneumonia, Roundworm—Baylisascariasis, Tapeworm—infection, Clonorchiasis, Dioctophyme renalis infection, Diphyllobothriasis—tapeworm, Guinea worm-Dracunculiasis, Echinococcosis—tapeworm, Pinworm—Enterobiasis, Liver fluke—Fasciolosi s, Fasciolopsiasis—intestinal fluke, Gnathostomiasis, Hymenolepiasis, Loa filariasis, Calabar swellings, Mansonelliasis, Filariasis, Metagonimiasis—intestinal fluke, River blindness, Chinese Liver Fluke, Paragonimiasis, Lung Fluke, Schistosomiasis-bilharzia, bilharziosis or snail fever (all types), intestinal schistosomiasis, urinary schistosomiasis, Schistosomiasis by Schistosoma japonicum, Asian intestinal schistosomiasis, Sparganosis, Strongyloidiasis—Parasitic pneumonia, Beef tapeworm, Pork tapeworm, Toxocariasis, Trichinosis, Swimmer's itch, Whipworm and Elephantiasis Lymphatic filariasis. The parasite may be an organism or disease caused by an organism such as, but not limited to, parasitic worm, Halzoun Syndrome, Myiasis, Chigoe flea, Human Botfly and Candiru. The parasite may be an ectoparasite or disease caused by an ectoparasite such as, but not limited to, Bedbug, Head louse-Pediculosis, Body louse-Pediculosis, Crab louse—Pediculosis, Demodex—Demodicosis, Scabies, Screwworm and Cochliomyia.

The antigen may be a protein derived from cancer. Tumor antigens are relatively restricted to tumor cells and can be any protein that induces an immune response. However, many tumor antigens are host (self) proteins and thus are typically not seen as antigenic by the host immune system. Tumor antigens can also be abnormally expressed by cancer cells. Tumor antigens can also be germline/testis antigens expressed in cancer cells, cell lineage differentiation antigens not expressed in adult tissue, or antigens overexpressed in cancer cells. The cancers, include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrim; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrm macroglobulinemia and Wilms tumor (kidney cancer.)

In some embodiments, the first heterologous antigen and second heterologous antigens are the same. In some embodiments, the first heterologous antigen and second heterologous antigens are different.

In some embodiments, the first CD8+ TCR recognizes specific MHC-II, MHC-E, or MHC-I subtopes or supertopes. In some embodiments, the first CD8+ TCR is identified by DNA or RNA sequencing. In some embodiments, the CD8+ TCR is identified by single cell sequencing.

In some embodiments, the one or more isolated CD8+ T cells from the second set of CD8+ T cells express CD69 and TNFα.

In some embodiments, the second CD8+ TCR recognizes one or more specific MHC-II supertopes, MHC-E supertopes, and/or MHC-I supertopes. In further examples, the second CD8+ TCR recognizes a MHC-II supertope and a MHC-E supertope, a WW-II supertope and a MHC-I supertope, or a MHC-I supertope and a MHC-E supertope.

In some embodiments, the second CD8+ TCR recognizes one or more specific MHC-II subtopes, MHC-E subtopes, and/or MHC-I subtopes. In further examples, second CD8+ TCR recognizes a MHC-II subtope and a MHC-E subtope, MHC-II subtope and a MHC-I subtope, or a MHC-I subtope and a MHC-E subtope.

In some embodiments, the second CD8+ TCR recognizes a MHC-II subtope or supertope and a MHC-E subtope or supertope, a MHC-II subtope or supertope and a MHC-I subtope or supertope, or a MHC-I subtope or supertope and a MHC-E subtope or supertope.

In some embodiments, the second CD8+ TCR recognizes specific MHC-II supertopes and MHC-II subtopes, supertopes and MHC-E subtopes, or MHC-I supertopes and MHC-I subtopes. In some embodiments, the second CD8+ TCR recognizes more than one MHC-II supertope from the same antigen, more than one supertope from the same antigen, or more than one MHC-I supertope from the same antigen. In some embodiments, the second CD8+ TCR recognizes more than one MHC-II subtope from the same antigen, more than one MHC-E subtope from the same antigen, or more than one MHC-I subtope from the same antigen.

In some embodiments, the second CD8+ TCR recognizes one or more MHC-II supertopes and one or more MHC-II subtopes from the same antigen, one or more MHC-E supertopes and one or more MHC-E subtopes from the same antigen, or one or more MHC-I supertopes and one or more MHC-I subtopes from the same antigen. In some embodiments, second CD8+ TCR recognizes more than one MHC-II supertope from more than one antigen, more than one MHC-E supertope from more than one antigen, or more than one MHC-I supertope from more than one antigen. In some embodiments, the second CD8+ TCR recognizes more than one MHC-II subtope from more than one antigen, more than one MHC-E subtope from more than one antigen, or more than one MHC-I subtope from more than one antigen.

In some embodiments, the second CD8+ TCR recognizes one or more MHC-II supertopes and one or more MHC-II subtopes from different antigens, one or more MEW-E supertopes and one or more MHC-E subtopes from different antigens, or one or more supertopes and one or more MHC-I subtopes from different antigens.

In some embodiments, the third CD8+ TCR recognizes one or more specific supertopes, MHC-E supertopes, or MHC-I supertopes. In some embodiments, the third CD8+ TCR recognizes one or more specific MHC-II subtopes, MHC-E subtopes, or MHC-I subtopes. In some embodiments, the third CD8+ TCR recognizes specific MHC-II supertopes and MHC-II subtopes, specific supertopes and subtopes, or specific MHC-I supertopes and MHC-I subtopes.

In some embodiments, the third CD8+ TCR recognizes more than one MHC-II supertope from one antigen, more than one supertope from one antigen, or more than one MHC-I supertope from one antigen. In some embodiments, the third CD8+ TCR recognizes more than one MHC-II subtope from one antigen, more than one subtope from one antigen, or more than one MHC-I subtope from one antigen.

In some embodiments, third CD8+ TCR recognizes one or more MHC-II supertopes and one or more MHC-II subtopes from one antigen, one or more supertopes and one or more MHC-E subtopes from one antigen, or one or more MHC-I supertopes and one or more MHC-I subtopes from one antigen. In some embodiments, the third CD8+ TCR recognizes specific subtopes or supertopes and MHC-II subtopes or supertopes, specific subtopes or supertopes and MHC-I subtopes or supertopes, or specific MHC-II subtopes or supertopes and MHC-I subtopes or supertopes.

In some embodiments, third CD8+ TCR recognizes third CD8+ TCR recognizes more than one MHC-II subtope from the same antigen, third CD8+ TCR recognizes more than one MHC-E subtope from the same antigen, or third CD8+ TCR recognizes more than one MHC-I subtope from the same antigen. In some embodiments, the third CD8+ TCR recognizes one or more MHC-II supertopes and one or more MHC-II subtopes from different antigens, one or more MHC-E supertopes and one or more subtopes from different antigens, or one or more MHC-I supertopes and one or more MHC-I subtopes from different antigens.

In some embodiments, the nucleic acid sequence encoding the third CD8+ TCR is identical to the nucleic acid sequence encoding the second CD8+ TCR.

In some embodiments, the method comprises isolating one or more CD8+ T cells from a second subject and transfecting the one or more CD8+ T cells with a nucleic acid sequence encoding the selected third CD8+ TCR and a promoter operably linked to the nucleic acid sequence encoding the third CD8+ TCR, thereby generating one or more CD8+ T cells that recognize the first MHC/heterologous antigen-derived peptide complex and the second MHC/heterologous antigen-derived peptide complex.

In some embodiments, the first MHC-heterologous antigen-derived peptide complex is a MHC-II/heterologous antigen-derived peptide complex, a MHC-E/heterologous antigen-derived peptide complex, or a MHC-I/heterologous antigen-derived peptide complex. In some embodiments, the second MHC-heterologous antigen-derived peptide complex is a MHC-II/heterologous antigen-derived peptide complex, a MHC-E/heterologous antigen-derived peptide complex, or a MHC-I/heterologous antigen-derived peptide complex.

In certain embodiments, CD8+ T cells comprising the multi-specific TCRs can be used for prevention or treatment of disease. The route of administration of the population of T cells and the amount to be administered to the human patient can be determined based on the condition of the human patient and the knowledge of the physician. In some embodiments, the route of administration is intravenous, intramuscular, intraperitoneal, or oral administration. Generally, the administration is intravenous.

In some embodiments, the CD8+ T cell is administered to treat or prevent cancer. In further examples, the cancer is prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, or cervical cancer.

In some embodiments, the CD8+ T cell is administered to treat or prevent a pathogenic infection. In further examples, the pathogenic infection is human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In certain embodiments, the administering is by infusion of the population of CD8+ T cells. In some embodiments, the infusion is bolus intravenous infusion. In certain embodiments, the administering comprises administering at least about 1×10⁵ T cells of the population of CD8+ T cells per kg per dose per week to the human patient. In certain embodiments, the administering comprises administering at least about 1×10⁶ T cells of the population of CD8+ T cells per kg per dose per week to the human patient.

In certain embodiments, the treatment methods comprise administering at least 2 doses of the population of CD8+ T cells to the human patient. In specific embodiments, the treatment methods comprise administering 2, 3, 4, 5, or 6 doses of the population of T cells to the human patient.

In some embodiments, wherein the first subject is a nonhuman primate and the second subject is a human, and wherein the transfected CD8+ T cells comprises a chimeric nonhuman primate-human CD8+ TCR comprising the non-human primate CDR3α and CDR3β of the second CD8+ TCR. In some embodiments, the third CD8+ TCR comprises the non-human primate CDR1α, CDR2α, CDR3α, CDR1β, CDR2β, and CDR3β of the second CD8+ TCR. In some embodiments, the third CD8+ TCR comprises the CDR1α, CDR2α, CDR3α, CDR1β, CDR2β, and CDR3β of the second CD8+ TCR. In some embodiments, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In some embodiments, wherein the third CD8+ TCR is a chimeric CD8+ TCR.

Also disclosed is a method of generating CD8+ T cells comprising a multi-specific T cell receptor (TCR), wherein the method comprises administering to a subject a recombinant CMV vector comprising a nucleic acid sequence that encodes a first heterologous antigen, in an amount effective to generate a first set of CD8+ T cells that recognize a first MHC-E/heterologous antigen-derived peptide complex, wherein the CMV vector does not express an active UL128, UL130, UL146 and UL147 protein or orthologs thereof and wherein the recombinant CMV vector further comprises a microRNA recognition element (MRE); identifying a first CD8+ TCR from the first set of CD8+ T cells, wherein the first CD8+ TCR recognizes the first MHC-E/heterologous antigen-derived peptide complex; administering to the subject a second heterologous antigen in an amount effective to generate a second set of CD8+ T cells that recognizes a second MHC-E/heterologous antigen-derived peptide complex; isolating one or more CD8+ T cells from the second set of CD8+ T cells; identifying a second CD8+ TCR from the second set of CD8+ T cells, wherein the second CD8+ TCR recognizes the first MHC-E/heterologous antigen-derived peptide complex and the second MHC-E/heterologous antigen-derived peptide complex; transfecting a third set of CD8+ T cells with an expression vector, wherein the expression vector comprises a nucleic acid sequence encoding a third CD8+ TCR and a promoter operably linked to the nucleic acid sequence encoding the third CD8+ TCR, wherein the third CD8+ TCR comprises CDR3α and CDR3β of the second CD8+ TCR, thereby generating one or more CD8+ T cells that recognize the first MHC-E/heterologous antigen-derived peptide complex and the second MHC-E/heterologous antigen-derived peptide complex; and selecting one or more of the third CD8+ TCRs with the highest avidity for a specific peptide of interest.

In some embodiments, the first heterologous antigen and second heterologous antigens are the same. In some embodiments, the first heterologous antigen and second heterologous antigens are different. In some embodiments, the subject is a human or non-human primate. In some embodiments, the recombinant CMV vector is a recombinant human CMV vector or a recombinant rhesus macaque CMV vector.

In some embodiments, the first heterologous antigen comprises a tumor antigen, pathogen-specific antigen, a tissue specific antigen, or a host-self antigen. In some embodiments, the tumor antigen is related to a cancer selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer. In some embodiments, pathogen-specific antigen is related to a pathogen selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In some embodiments, the MRE contains target sites for microRNAs expressed in endothelial cells. In further embodiments, the MRE is specific for the miRNA selected from the group consisting of miR126, miR-126-3p, miR-130a, miR-210, miR-221/222, miR-378, miR-296, and miR-328.

In some embodiments, the first CD8+ TCR recognizes specific MHC-E subtopes or supertopes. In some embodiments, the first CD8+ TCR is identified by DNA or RNA sequencing. In some embodiments, the CD8+ TCR is identified by single cell sequencing.

In some embodiments, the one or more isolated CD8+ T cells from the second set of CD8+ T cells express CD69 and TNFα.

In some embodiments, the second CD8+ TCR recognizes one or more specific MHC-E supertopes. In some embodiments, the second CD8+ TCR recognizes one or more specific MHC-E subtopes. In some embodiments, the second CD8+ TCR recognizes specific MHC-E supertopes and MHC-E subtopes.

In some embodiments, the second CD8+ TCR recognizes more than one MHC-E supertope from the same antigen. In some embodiments, the second CD8+ TCR recognizes more than one MHC-E subtope from the same antigen.

In some embodiments, the second CD8+ TCR recognizes one or more MHC-E supertopes and one or more MHC-E subtopes from the same antigen. In some embodiments, the second CD8+ TCR recognizes more than one MHC-E subtope from more than one antigen.

In some embodiments, the second CD8+ TCR recognizes one or more MHC-E supertopes and one or more MHC-E subtopes from different antigens.

In some embodiments, the third CD8+ TCR recognizes one or more specific MHC-E supertopes. In some embodiments, the third CD8+ TCR recognizes one or more specific MHC-E subtopes. In some embodiments, the third CD8+ TCR recognizes specific MHC-E supertopes and MHC-E subtopes.

In some embodiments, the third CD8+ TCR recognizes more than one MHC-E supertope from one antigen. In some embodiments, the third CD8+ TCR recognizes more than one MHC-E subtope from one antigen.

In some embodiments, third CD8+ TCR recognizes one or more MHC-E supertopes and one or more MHC-E subtopes from one antigen.

In some embodiments, third CD8+ TCR recognizes third CD8+ TCR recognizes more than one MHC-E subtope from the same antigen. In some embodiments, the third CD8+ TCR recognizes one or more MHC-E supertopes and one or more MHC-E subtopes from different antigens.

In some embodiments, the nucleic acid sequence encoding the third CD8+ TCR is identical to the nucleic acid sequence encoding the second CD8+ TCR.

In some embodiments, the method comprises isolating one or more CD8+ T cells from a second subject and transfecting the one or more CD8+ T cells with a nucleic acid sequence encoding the selected third CD8+ TCR and a promoter operably linked to the nucleic acid sequence encoding the third CD8+ TCR, thereby generating one or more CD8+ T cells that recognize the first MHC-E/heterologous antigen-derived peptide complex and the second MHC-E/heterologous antigen-derived peptide complex.

In certain embodiments, CD8+ T cells comprising the multi-specific TCRs can be used for prevention or treatment of disease. The route of administration of the population of T cells and the amount to be administered to the human patient can be determined based on the condition of the human patient and the knowledge of the physician. In some embodiments, the route of administration is intravenous, intramuscular, intraperitoneal, or oral administration. Generally, the administration is intravenous.

In some embodiments, the CD8+ T cell is administered to treat or prevent cancer. In further examples, the cancer is prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, or cervical cancer.

In some embodiments, the CD8+ T cell is administered to treat or prevent a pathogenic infection. In further examples, the pathogenic infection is human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

In certain embodiments, the administering is by infusion of the population of CD8+ T cells. In some embodiments, the infusion is bolus intravenous infusion. In certain embodiments, the administering comprises administering at least about 1×10⁵ T cells of the population of CD8+ T cells per kg per dose per week to the human patient. In certain embodiments, the administering comprises administering at least about 1×10⁶ T cells of the population of CD8+ T cells per kg per dose per week to the human patient.

In certain embodiments, the treatment methods comprise administering at least 2 doses of the population of CD8+ T cells to the human patient. In specific embodiments, the treatment methods comprise administering 2, 3, 4, 5, or 6 doses of the population of T cells to the human patient.

In some embodiments, wherein the first subject is a nonhuman primate and the second subject is a human, and wherein the transfected CD8+ T cells comprises a chimeric nonhuman primate-human CD8+ TCR comprising the non-human primate CDR3α and CDR3β of the second CD8+ TCR. In some embodiments, the third CD8+ TCR comprises the non-human primate CDR1α, CDR2α, CDR3α, CDR1β, CDR2β, and CDR3β of the second CD8+ TCR. In some embodiments, the third CD8+ TCR comprises the CDR1α, CDR2α, CDR3α, CDR1β, CDR2β, and CDR3β of the second CD8+ TCR. In some embodiments, the first subject is a nonhuman primate and the second subject is a human, and wherein the second CD8+ TCR is a chimeric nonhuman primate-human CD8+ TCR comprising the non-human primate CDR3α and CDR3β of the first CD8+ TCR. In some embodiments, wherein the third CD8+ TCR is a chimeric CD8+ TCR.

The multi-specific TCRs disclosed herein may be used in methods of inducing an immunological response in a subject comprising administering to the subject a composition comprising a CD8+ T cell comprising the multi-specific TCR and a pharmaceutically acceptable carrier or diluent. For purposes of this specification, the term “subject” includes all animals, including non-human primates and humans, while “animal” includes all vertebrate species, except humans; and “vertebrate” includes all vertebrates, including animals (as “animal” is used herein) and humans. And, of course, a subset of “animal” is “mammal”, which for purposes of this specification includes all mammals, except humans.

As to antigens for use in vaccine or immunological compositions, see also Stedman's Medical Dictionary (24th edition, 1982, e.g., definition of vaccine (for a list of antigens used in vaccine formulations); such antigens or epitopes of interest from those antigens may be used. As to tumor antigens, one skilled in the art may select a tumor antigen and the coding DNA therefor from the knowledge of the amino acid and corresponding DNA sequences of the peptide or polypeptide, as well as from the nature of particular amino acids (e.g., size, charge, etc.) and the codon dictionary, without undue experimentation.

A wide variety of appropriate host cells may be used to express the multi-specific TCR of the invention, including but not limited to mammalian cells (animal cells) plant: cells, bacteria (e.g. Bacillus subtilis, Escherichia coli), insect cells, and yeast (e.g. Pichia pastoris, Saccharomyces cerevisiae). For example, a variety of cell lines that may find use in the present invention are described in the ATCC cell line catalog, available from the American Type Culture Collection. Furthermore, also plants and animals may be used as hosts for the expression of the T-cell receptor according to the present invention. The expression as well as the transfection vectors or cassettes may be selected according to the host used.

Of course also non-cellular or cell-free protein expression systems may be used. In vitro transcription/translation protein expression platforms, that produce sufficient amounts of protein offer many advantages of a cell-free protein expression, eliminating the need for laborious up- and down-stream steps (e.g. host cell transformation, culturing, or lysis) typically associated with cell-based expression systems.

An immune response to a tumor antigen is generated, in general, as follows: T cells recognize proteins only when the protein has been cleaved into smaller peptides and is presented in a complex called the “major histocompatibility complex (MHC)” located on another cell's surface. There are two classes of MHC complexes—class I and class II, and each class is made up of many different alleles. Different species, and individual subjects have different types of MHC complex alleles; they are said to have a different MEW type. One type of MEW class I molecule is called MHC-E (HLA-E in humans, Mamu-E in RM, Qa-lb in mice). Unlike other MHC-I molecules, MHC-E is highly conserved within and between mammalian species.

Further disclosed are pharmaceutical and other compositions containing the disclosed multi-specific TCRs. Such pharmaceutical and other compositions may be formulated so as to be used in any administration procedure known in the art. Such pharmaceutical compositions may be via a parenteral route (intradermal, intramuscular, subcutaneous, intravenous, or others). The administration may also be via a mucosal route, e.g., oral, nasal, genital, etc.

The disclosed pharmaceutical compositions may be prepared in accordance with standard techniques well known to those skilled in the pharmaceutical arts. Such compositions may be administered in dosages and by techniques well known to those skilled in the medical arts taking into consideration such factors as the breed or species, age, sex, weight, and condition of the particular patient, and the route of administration. The compositions may be administered alone, or may be co-administered or sequentially administered with other with other immunological, antigenic or therapeutic compositions.

The disclosed CMV vectors may be administered in vivo, for example where the aim is to produce an immunogenic response, including a CD8+ immune response, including an immune response characterized by a high percentage of the CD8+ T cell response being restricted by MHC-E, MHC-II, or MHC-I (or a homolog or ortholog thereof). For example, in some examples it may be desired to use the disclosed CMV vectors in a laboratory animal, such as rhesus macaques for preclinical testing of immunogenic compositions and vaccines using RhCMV. In other examples, it will be desirable to use the disclosed CMV vectors in human subjects, such as in clinical trials and for actual clinical use of the immunogenic compositions using HCMV.

For such in vivo applications the disclosed CMV vectors are administered as a component of an immunogenic composition further comprising a pharmaceutically acceptable carrier. In some embodiments, the immunogenic compositions of the disclosure are useful to stimulate an immune response against the heterologous antigen, including a tumor antigen, a tumor virus antigen, or a host self-antigen and may be used as one or more components of a prophylactic or therapeutic vaccine against tumor antigens, tumor virus antigens, or host self-antigens for the prevention, amelioration or treatment of cancer. The nucleic acids and vectors of the disclosure are particularly useful for providing genetic vaccines, i.e., vaccines for delivering the nucleic acids encoding the antigens of the disclosure to a subject, such as a human, such that the antigens are then expressed in the subject to elicit an immune response.

Immunization schedules (or regimens) are well known for animals (including humans) and may be readily determined for the particular subject and immunogenic composition. Hence, the immunogens may be administered one or more times to the subject. Preferably, there is a set time interval between separate administrations of the immunogenic composition. While this interval varies for every subject, typically it ranges from 10 days to several weeks, [and is often 2, 4, 6 or 8 weeks. For humans, the interval is typically from 2 to 6 weeks. In a particularly advantageous embodiment of the present disclosure, the interval is longer, advantageously about 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks, 54 weeks, 56 weeks, 58 weeks, 60 weeks, 62 weeks, 64 weeks, 66 weeks, 68 weeks or 70 weeks. The immunization regimes typically have from 1 to 6 administrations of the immunogenic composition, but may have as few as one or two or four. The methods of inducing an immune response may also include administration of an adjuvant with the immunogens. In some instances, annual, biannual or other long interval (5-10 years) booster immunization may supplement the initial immunization protocol. The present methods also include a variety of prime-boost regimens. In these methods, one or more priming immunizations are followed by one or more boosting immunizations. The actual immunogenic composition may be the same or different for each immunization and the type of immunogenic composition (e.g., containing protein or expression vector), the route, and formulation of the immunogens may also be varied. For example, if an expression vector is used for the priming and boosting steps, it may either be of the same or different type (e.g., DNA or bacterial or viral expression vector). One useful prime-boost regimen provides for two priming immunizations, four weeks apart, followed by two boosting immunizations at 4 and 8 weeks after the last priming immunization. It should also be readily apparent to one of skill in the art that there are several permutations and combinations that are encompassed using the DNA, bacterial and viral expression vectors of the disclosure to provide priming and boosting regimens. CMV vectors may be used repeatedly while expressing different antigens derived from different pathogens.

### EXAMPLES

### Example 1: TCR Clonotype Hierarchies of MHC-E Supertope Responses

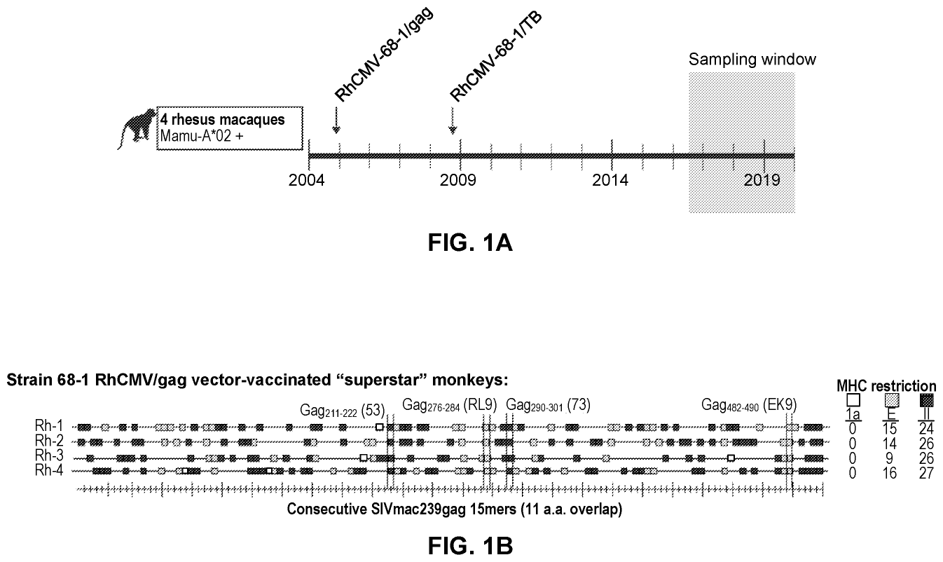

On average, after vaccination with RhCMV vectors lacking functional expression of the RhCMV homologues of human CMV UL128, UL130, UL146 and UL147 while expressing the homologs of UL40 and US28, —1 Mamu E-restricted CD8+ T cell epitope (typically 99mers) per every 30-40 amino acids of protein length can be identified. Responses to some of these epitopes are shared by all monkeys and these epitopes are referred to as supertopes. For example, four animals inoculated with strain 68-1 RhCMV expressing the antigen SIVgag elicit T cell responses to the MHC-E supertopes gag69 and gag120 (FIGS. 1A-1B) (strain 68-1 spontaneously acquired above mentioned genetic modifications).

The characterization of the TCRs responsible for the SIV-specific CD8+ T cell responses from these 4 long-term 68-1 RhCMV/SIVgag vector vaccinated rhesus monkeys (RM) that developed long-standing and well-characterized unconventionally restricted, SIVgag-specific CD8+ T cell responses that were elicited and maintained by 68-1 RhCMV/SIVgag vaccination in these animals over the past 15 years is shown in FIGS. 1A-1B. These RM were also used to test a 68-1 RhCMV/TB vector expressing the ESAT-6 and Ag85B TB antigens about 4 years after RhCMV/SIVgag vaccination.

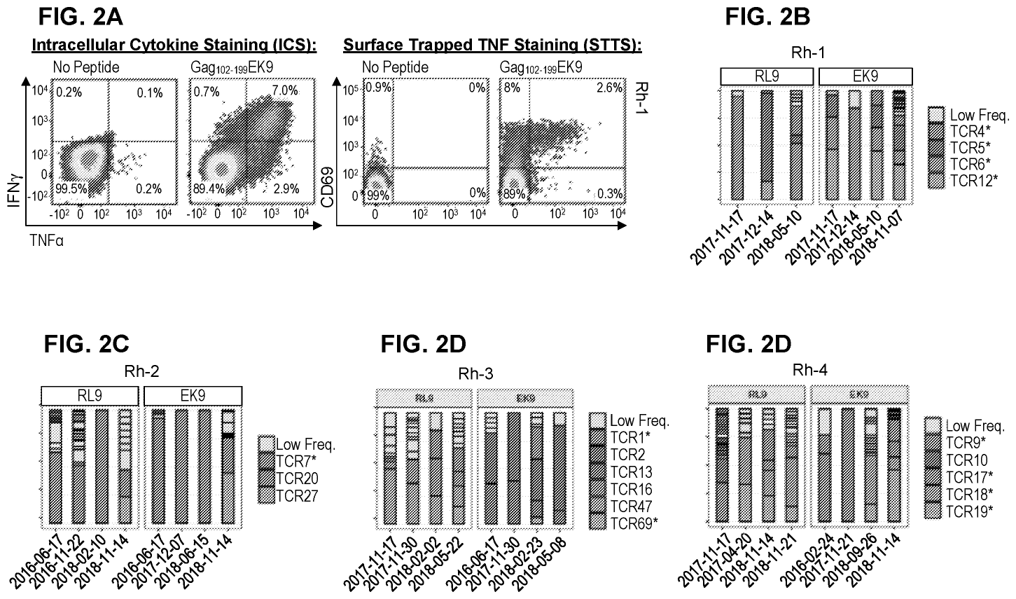

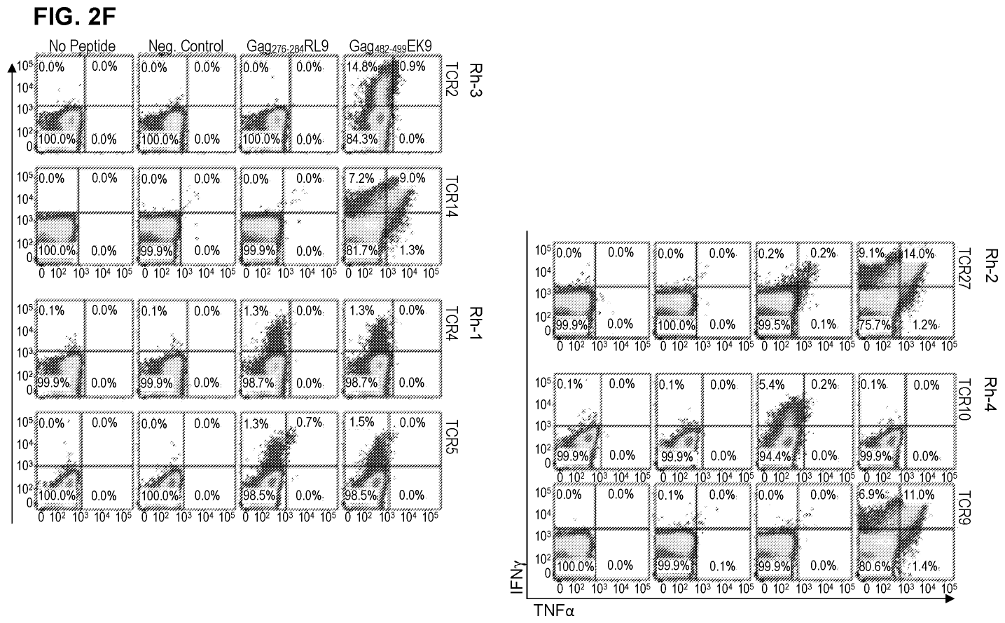

Using surface-trapped TNF staining (STTS; FIG. 2A), viable (non-fixed) epitope-responsive CD8+ T cells were able to be sorted from these RM that are suitable for single cell (sc) transcriptomic analysis, including sequencing of all expressed TCR chains and overall analysis of each cell's transcriptome. The characterization of the TCR clonotypic structure of the 68-1 RhCMV/SIV vector-elicited CD8+ T cell responses to the two MHC-E-restricted SIVgag supertopes, Gag₂₇₆₋₂₈₄ RL9 (Gag69) and Gag₄₈₂₋₄₉₉ EK9 (Gag120) by analysis of sorted CD8+ T cells that respond to these epitopes with both CD69 upregulation and surface-trapped TNF expression was analyzed (this double positivity criterion used for maximum specificity; of note, though, since not all responding clonotypes express detectable stTNF, responding clonotype+ cells can also be the CD69+/stTNF− fraction; see comparison of parallel ICS and STTS assays in FIG. 2A). STTS was used longitudinally over a three-year period to sort EK9- and RL9-specific T cells (stTNF+/sCD69+) for each study RM. Sorted cells were analyzed by bulk- and/or single-cell RNAseq, allowing identification of their complete TCR α/β hierarchies. Strikingly, when Gag supertope-responding CD8+ T cells were analyzed for TCR expression, each of the 4 study RM manifested a stable highly oligoclonal clonotypic hierarchy for both Gag276-284RL9 and Gag482-499EK9, and unexpectedly, many clonotypes were shared across these 2 supertope-specific responses (despite the fact that the 2 supertope optimal 9mer peptides have essentially no sequence homology; FIGS. 2B-2E).

### Example 2: Some MHC-E Restricted Cd8+ TCRs Recognize Sequence-Unrelated Supertopes and Endogenously Processed Antigen

Each of the major TCR alpha/beta chain pairs from all four RM were cloned for specificity analysis using transduction of primary control (SIV Ag naïve) RM CD8+ T cells. As shown in FIG. 2F, each scRNAseq-identified TCR mediated a specific response to Gag₂₇₆₋₂₈₄ RL9, Gag₄₈₂₋₄₉₉ EK9 or both, confirming the specificities revealed by scRNAseq and unequivocally demonstrating that individual TCRs can have dual specificity to these 2 MHC-E-restricted supertopes.

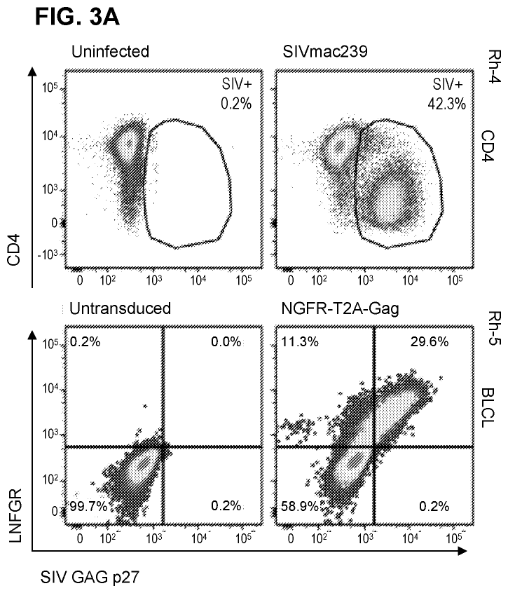

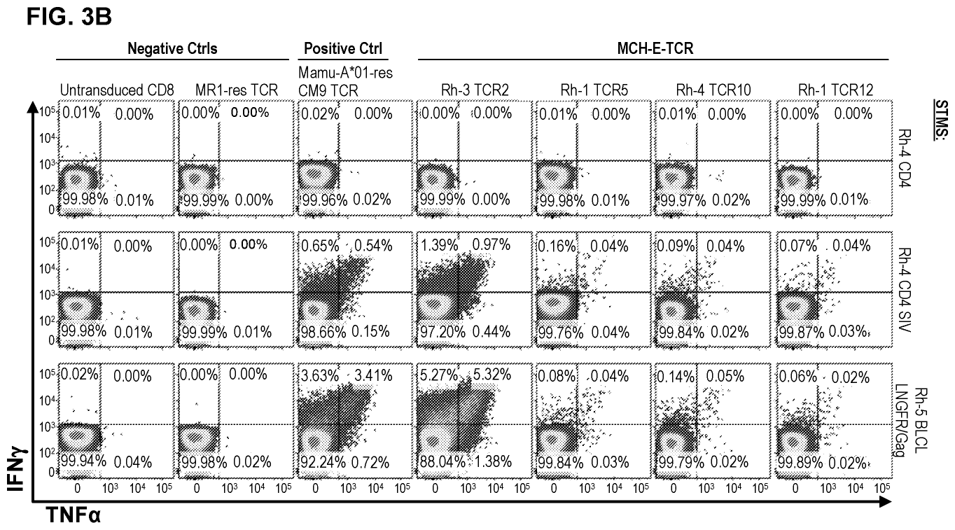

It was also demonstrated that these TCRs can specifically recognize SIVmac239-infected CD4+ T cells and B lymphoblastoid cell lines (BLCL) transfected with SIVgag (FIGS. 3A-3B), demonstrating that SIVgag epitopic peptides can be effectively processed and surface expressed in the context of MHC-E in non-RhCMV-infected cells.

### Example 4: The Broad Epitope Specificity of the MHC-E Restricted CD8+ T Cell Response is Mediated by a Small Number of TCRs

As shown in FIG. 1A, the SIVgag-specific CD8+ T cells maintained in these 4 RM recognize a minimum of 9-16 different MHC-E-restricted epitopes and 23-27 MHC-II-restricted epitopes. To identify the full extent of the TCR clonotypes in these RM that can recognize naturally processed SIVgag in SIV-infected cells, CD8+ T cells from each study RM were stimulated with autologous SIV-infected CD4+ T cells, identified responding cells by STTS, sorted the responding cells on the basis of sCD69 and stTNF, and then analyzed the responding cells by scRNAseq, as shown in FIG. 2.

To determine the clonotypic hierarchies for SIV-infected recognition, purified CD8+ T cells from each study RM were incubated with autologous SIV-infected CD4s. Activated cells were sorted based on sCD69 and s-tTNF-α staining, followed by single-cell RNA-seq. Pie charts illustrate the relative frequency of each clone. Additionally, clones identified in >5% of responding cells in at least 2 separate supertope peptide stims, but present at <5% in this experiment are also named. The clone name, alpha/beta CDR3 sequences, and V/J segment usage are shown in Tables 1-4.

TABLE 1
Clone name, alpha/beta CDR3 sequences, and V/J segment used with Rh-3.

| | TRA CDR3 (SEQ | | | |
|---|---|---|---|---|
| | ID NO) | TRA V/J | TRB CDR3 (SEQ ID NO) | TRB V/J |
| TCR1 | CAGRDNFNKFY | TRAV1- | CASSPREDANYDYTF | TRBV27/TRB |
| -1 | F (SEQ ID NO: 1) | 2/TRAJ22 | (SEQ ID NO: 2) | J1-2 |
| TCR1 | CARPDSGWQLT | TRAV1- | CASSPREDANYDYTF | TRBV27/TRB |
| -2 | F (SEQ ID NO: 3) | 2/TRAJ22 | (SEQ ID NO: 4) | J1-2 |
| TCR2 | CAGLGVASNKL | TRAV35/TRA | CASSYSLKNTQYF (SEQ | TRBV6- |
| | TF (SEQ ID NO: | J17 | ID NO: 6) | 3/TRBJ2-4 |
| | 5) | | | |
| TCR1 | CALSNSGYSTLT | TRAV16/TRA | CASRKDRSEQYF (SEQ ID | TRBV6- |
| 3-1 | F (SEQ ID NO: 7) | J11 | NO: 8) | 8/TRBJ2-7 |
| TCR1 | CASNSGWQLTF | TRAV8- | CASRKDRSEQYF (SEQ ID | TRBV6- |
| 3-2 | (SEQ ID NO: 9) | 7/TRAJ22 | NO: 10) | 8/TRBJ2-7 |
| TCR1 | CLVRTGNVLTF | TRAV4/TRAJ | CASSPQDRVETQYF (SEQ | TRBV5- |
| 4 | (SEQ ID NO: 11) | 39 | ID NO: 12) | 9/TRBJ2-5 |
| TCR1 | CAVESDTGGFK | TRAV22- | CASSQEVGGGGVENTQY | TRBV4- |
| 6 | TVF (SEQ ID NO: | 1/TRAJ9 | F (SEQ ID NO: 14) | 3/TRBJ2-4 |
| | 13) | | | |
| TCR4 | CMDTGIASKLTF | TRAV26- | CASSFYLWGSSGASVLTF | TRBV7- |
| 7 | (SEQ ID NO: 15) | 1/TRAJ44 | (SEQ ID NO: 16) | 4/TRBJ2-6 |
| TCR6 | CLVGTGIASKLT | TRAV4/TRAJ | CASSYRDRAETQYF (SEQ | TRBV5- |
| 8 | F (SEQ ID NO: | 44 | ID NO: 18) | 5/TRBJ2-5 |
| | 17) | | | |
| TCR6 | CAGRSSYNKLM | TRAV25/TRA | CASSPREDSNYDYTF | TRBV27/TRB |
| 9 | F (SEQ ID NO: | J50 | (SEQ ID NO:20) | J1-2 |
| | 19) | | | |

TABLE 2
Clone name, alpha/beta CDR3 sequences, and V/J segment used with Rh-1.

| | TRA CDR3 (SEQ ID | | TRB CDR3 (SEQ ID | |
|---|---|---|---|---|
| | NO) | TRA V/J | NO) | TRB V/J |
| TCR4 | CAGRDNFNKFYF | TRAV1- | CASSFRDDANYDY | TRBV27/TRBJ |
| | (SEQ ID NO:21) | 2/TRAJ22 | TF (SEQ ID NO: 22) | 1-2 |
| TCR5 | CALRDLRNSGNRAL | TRDV1/TRAJ2 | CASSPGLGEEETQY | TRBV11- |
| | VF (SEQ ID NO: 23) | 9 | F (SEQ ID NO: 24) | 2/TRBJ2-5 |
| TCR6 | CGAEIEDGQKLLF | TRAV30/TRAJ | CASSYSGINTQYF | TRBV6- |
| -1 | (SEQ ID NO: 25) | 16 | (SEQ ID NO: 26) | 3/TRBJ2-4 |
| TCR6 | CAVYGNKLIF (SEQ | TRAV8- | CASSYSGINTQYF | TRBV6- |
| -2 | ID NO: 27) | 2/TRAJ47 | (SEQ ID NO: 28) | 3/TRBJ2-4 |
| TCR1 | CALRELLGSGNRAL | TRDV1/TRAJ2 | CASSEVGEENTQY | TRBV6- |
| 2 | VF (SEQ ID NO: 29) | 9 | F (SEQ ID NO: 30) | 1/TRBJ2-4 |

TABLE 3
Clone name, alpha/beta CDR3 sequences, and V/J segment used with Rh-2

| | TRA CDR3 (SEQ ID | | TRB CDR3 (SEQ ID | |
|---|---|---|---|---|
| | NO) | TRA V/J | NO) | TRB V/J |
| TCR | CAGRAGRGSTLGK | TRAV25/TRAJ18 | CASSRSEGVTLGAD | TRBV5- |
| 7 | LYF (SEQ ID NO: | | PQYF (SEQ ID NO: | 1/TRBJ2-3 |
| | 31) | | 32) | |
| TCR | CVLIHGNKLIF | TRAV18/TRAJ47 | CVAGGGGNTAQLFF | TRBV28/TR |
| 20 | (SEQ ID NO: 33) | | (SEQ ID NO: 34) | BJ2-2 |
| TCR | CASMDSNYQLIW | TRAV4/TRAJ11 | CASSQEGIGTGGNA | TRBV4- |
| 21 | (SEQ ID NO: 35) | | QLFF (SEQ ID NO: | 2/TRBJ2-2 |
| | | | 36) | |
| TCR | CLVGDRRYSTLTF | TRAV4/TRAJ11 | CASSFRDRAETQYF | TRBV5- |
| 27 | (SEQ ID NO: 37) | | (SEQ ID NO: 38) | 6/TRBJ2-5 |
| TCR | CLVRTGGFKTVF | TRAV4*01/TRAJ | CASSFRDRQETQYF | TRBV5- |
| 28 | (SEQ ID NO: 39) | 9*01 | (SEQ ID NO: 40) | 8/TRBJ2-5 |

TABLE 4
Clone name, alpha/beta CDR3 sequences, and V/J segment used with Rh-4.

| | TRA CDR3 (SEQ ID | | TRB CDR3 (SEQ ID | |
|---|---|---|---|---|
| | NO) | TRA V/J | NO) | TRB V/J |
| TCR9 | CIVRRASGGGYVL | TRAV26- | CASSEGVLAGYDYT | TRBV6- |
| | TF (SEQ ID NO: 41) | 1/TRAJ6 | F (SEQ ID NO: 42) | 1/TRBJ1-2 |
| TCR1 | CAVNAGQAGTALI | TRAV8- | CASSLFFQEGTAQL | TRBV27/TRBJ |
| 0 | F (SEQ ID NO: 43) | 2/TRAJ15 | FF (SEQ ID NO: 44) | 2-2 |
| TCR1 | CALRERFGNEKLTF | TRDV1/TRAJ4 | CASSLDGGRYDYTF | TRBV27/TRBJ |
| 7 | (SEQ ID NO: 45) | 8 | (SEQ ID NO: 46) | 1-2 |
| TCR1 | CALWELGNTGKLI | TRDV1/TRAJ3 | CASSLVEGNTQYF | TRBV5- |
| 8 | F (SEQ ID NO: 47) | 7 | (SEQ ID NO: 48) | 10/TRBJ2-4 |
| TCR1 | CLLRDSGYSTLTF | TRAV4/TRAJ1 | CASSYRDRQETQYF | TRBV5- |
| 9 | (SEQ ID NO: 49) | 1 | (SEQ ID NO: 50) | 9/TRBJ2-5 |

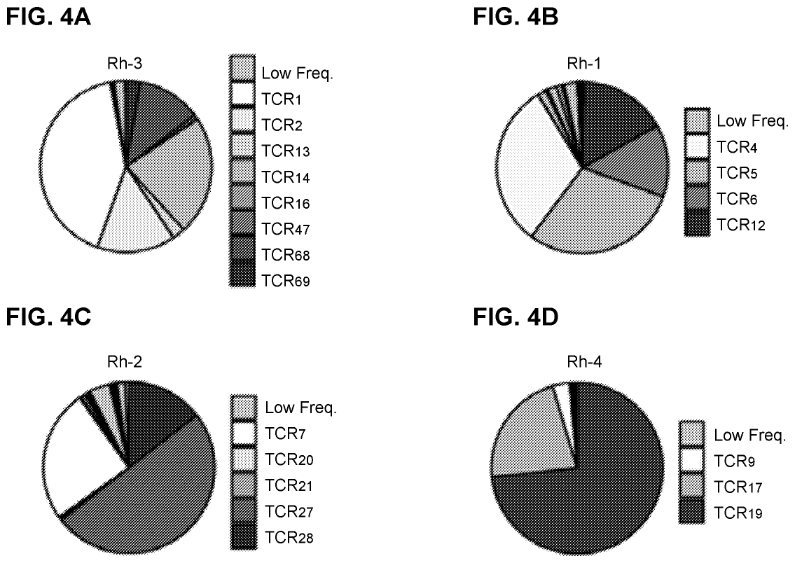

Of note, several clones that encoded two alpha chains and one beta (TCR1-1/2, TCR6-1/2, and TCR13-1/2; highlighted in grey) were identified. While no clonotypes were completely shared between RM, there is one alpha chain shared between Rh-3 and Rh-1 (FIGS. 4A and 4B, TCR1-1 and TCR4; red).

Strikingly, the TCR hierarchies of the CD8+ T cells responding to SIV-infected cells were highly oligoclonal and comprised almost entirely (90%+) by TCRs previously identified by MHC-E-restricted supertope responsiveness (FIGS. 4A-4D). With the exception of one TCR alpha chain shared by Rh-3 and Rh-1, these TCRs were distinct in each RM. Of note, two clonotypes in Rh-3 and one in Rh-1 expressed two TCR alphas chains, resulting the in the potential for these cells to express two distinct TCR, in which the beta chain pairs with either alpha chain.

### Example 5: MHC-E Restricted TCRs Recognizing Multiple, Unrelated Peptides

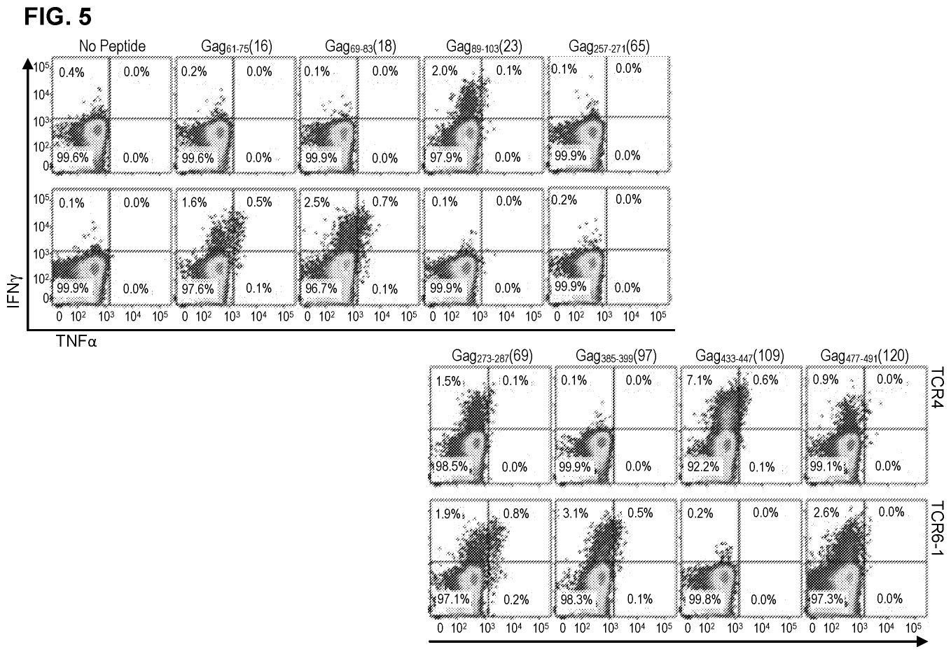

These data raise the question of where the TCRs that recognize the non-supertope epitopes (what is termed “subtopes”) are in these SIV-infected cell recognition assays, and suggest that either these subtopes are not processed or MHC-E-presented in SIV-infected cells (e.g., implying that only the supertopes are appropriately processed/presented, a rather unlikely possibility) or that the supertope-responsive TCR, already shown to be often cross-reactive between supertopes, also cross-react with subtopes. Indeed, as shown in FIG. 5, this latter possibility is the case.

MHC-E-TCR CD8+ T cell transductants from the overall 4 RM study cohort were tested as shown in FIG. 5 against a panel of MHC-E-restricted optimal peptides that were recognized in any of the study RM. For an epitope to be regarded as positive, it must have stimulated responses of >0.3% above background in >2 independent assays. The overall pattern of response for each TCR is shown in Tables 5-8 (note: not all targeted MHC-E-presented peptides trigger in all assays). ND: no data (analysis pending).

TABLE 5
Analysis of epitope cross-reactivity using TCR transductants in Rh-3.

| | Gag61- | Gag69- | Gag89- | Gag117- | Gag 129- | Gag 197- | Gag257- | Gag273- | Gag385- | Gag433- | Gag473- | Gag477- |
|---|---|---|---|---|---|---|---|---|---|---|---|---|
| | 75(16) | 83(18) | 103(23) | 131(30) | 143(33) | 211(50) | 271(65) | 287(69) | 399(97) | 447(109) | 487(119) | 491(120) |
| TCR1-1 | − | + | − | + | + | + | + | − | − | + | − | + |
| TCR1-2 | ND | ND | ND | ND | ND | ND | ND | ND | ND | ND | ND | ND |
| TCR2 | − | + | + | + | + | + | + | + | − | + | + | + |
| TCR13-1 | − | − | − | − | − | − | + | + | − | − | − | + |
| TCR13-2 | ND | ND | ND | ND | ND | ND | ND | ND | ND | ND | ND | ND |
| TCR14 | − | − | − | − | − | − | − | − | − | − | − | + |
| TCR16 | − | + | + | + | − | + | − | + | − | − | − | − |
| TCR47 | − | − | − | − | − | − | − | + | − | − | − | − |
| TCR68 | ND | ND | ND | ND | ND | ND | ND | ND | ND | ND | ND | ND |
| TCR69 | ND | ND | ND | ND | ND | ND | ND | ND | ND | ND | ND | ND |

TABLE 6
Analysis of epitope cross-reactivity using TCR transductants in Rh-1.

| | Gag61- | Gag69- | Gag89- | Gag117- | Gag129- | Gag197- | Gag257- | Gag273- | Gag385- | Gag433- | Gag473- | Gag477- |
|---|---|---|---|---|---|---|---|---|---|---|---|---|
| | 75(16) | 83(18) | 103(23) | 131(30) | 143(33) | 211(50) | 271(65) | 287(69) | 399(97) | 447(109) | 487(119) | 491(120) |
| TCR4 | − | − | + | + | + | − | − | + | − | + | − | + |
| TCR5 | − | − | − | − | − | − | − | + | − | − | − | + |
| TCR6-1 | + | + | + | + | + | + | − | + | + | + | + | + |
| TCR6-2 | − | + | + | − | − | + | − | + | + | + | + | + |
| TCR12 | ND | ND | ND | ND | ND | ND | ND | ND | ND | ND | ND | ND |

TABLE 7
Analysis of epitope cross-reactivity using TCR transductants in Rh-2.

| | Gag61- | Gag69- | Gag89- | Gag117- | Gag129- | Gag 197- | Gag257- | Gag273- | Gag385- | Gag433- | Gag473- | Gag477- |
|---|---|---|---|---|---|---|---|---|---|---|---|---|
| | 75(16) | 83(18) | 103(23) | 131(30) | 143(33) | 211(50) | 271(65) | 287(69) | 399(97) | 447(109) | 487(119) | 491(120) |
| TCR7 | + | − | + | + | + | − | + | + | + | + | + | + |
| TCR20 | − | + | + | − | − | − | + | + | − | + | + | − |
| TCR21 | ND | ND | ND | ND | ND | ND | ND | ND | ND | ND | ND | ND |
| TCR27 | + | − | − | − | + | − | − | + | − | + | − | + |
| TCR28 | ND | ND | ND | ND | ND | ND | ND | ND | ND | ND | ND | ND |

TABLE 8
Analysis of epitope cross-reactivity using TCR transductants in Rh-4.

| | Gag61- | Gag69- | Gag89- | Gag117- | Gag 129- | Gag 197- | Gag257- | Gag273- | Gag385- | Gag433- | Gag473- | Gag477- |
|---|---|---|---|---|---|---|---|---|---|---|---|---|
| | 75(16) | 83(18) | 103(23) | 131(30) | 143(33) | 211(50) | 271(65) | 287(69) | 399(97) | 447(109) | 487(119) | 491(120) |
| TCR9 | ND | ND | ND | ND | ND | ND | ND | ND | ND | ND | ND | ND |
| TCR10 | − | − | − | − | − | − | − | + | − | − | − | − |
| TCR17 | − | − | + | + | − | − | + | + | + | + | − | + |
| TCR18 | + | + | + | + | + | + | + | + | + | + | + | + |

Of the 17 supertope-reactive TCR examined for reactivity to 10 of the most common subtopes, 12 of these TCRs showed cross-reactivity with at least 1 and up to all 10 of these subtopes (the other 5 TCRs only showing reactivity to one or both supertopes only, although cross-reactivity with other non-tested subtopes can't be ruled out). These data unequivocally demonstrate the MHC-E-restricted CD8+ T cell responses elicited and maintained by 68-1 RhCMV/SIVgag vectors predominantly use TCRs that are multi-specific, suggesting that the majority of the TCR clonotypes comprising the vaccine-elicited responses in these RM have the potential to recognize multiple distinct MHC-E-presented epitopes on the surface of SIV-infected target cells.

### Example 6: Generation of MHC-E Restricted TCRs Recognizing More than One Antigen

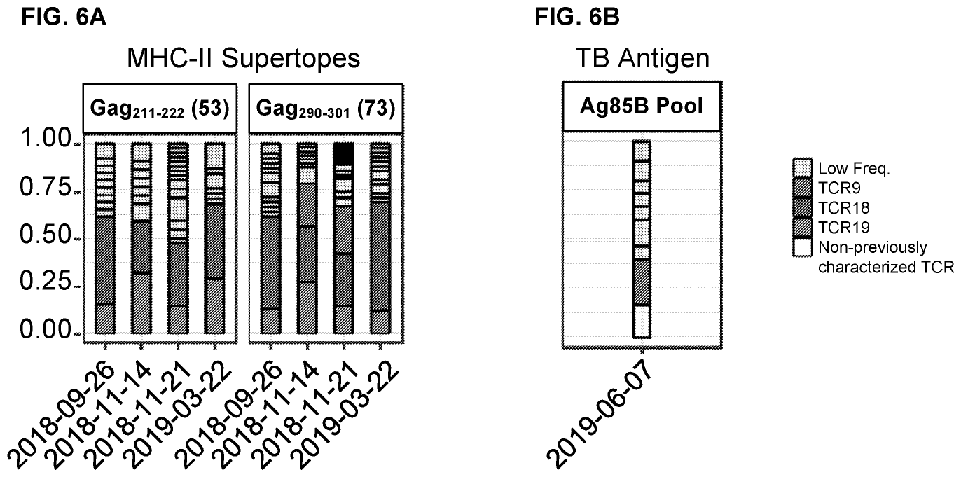

As shown in FIG. 1A, all RM in this study cohort were previously vaccinated with 68-1 RhCMV/TB vectors expressing an ESAT-6/Ag 85B polyprotein. ScRNA analysis of the CD8+ T cell response to an Ag85B peptide mix reveals that at least one TCR previously characterized as SIVgag-specific (TCR9) also responds to an epitope within this heterologous Ag (FIG. 6A; TCR6 from Rh33034 also appears to have a similar cross-reactivity). Three of the five dominant clonotypes previously identified by their MHC-E restricted SIVgag reactivity also respond to one or both of the MHC-II-restricted SIVgag supertope peptides, and one of these TCR (TCR9) also responds to a TB Ag85B epitope (FIG. 6B).

Thus, by an as yet uncharacterized mechanism, RM vaccinated with 68-1 RhCMV vectors develop responses that are Ag-targeted by highly cross-reactive TCRs, with the cross-reactivity not only involving MHC-E-presented epitopes within a particular Ag insert, but also MHC-E-restricted epitopes within a heterologous insert expressed by a 68-1 RhCMV-based vaccine that was administered at a different time.

### Example 7: Some MHC-E Restricted, SIVGAG-Specific TCRs are Derived from MHC-IA-Restricted, RhCMV-IE1 Specific TCRs

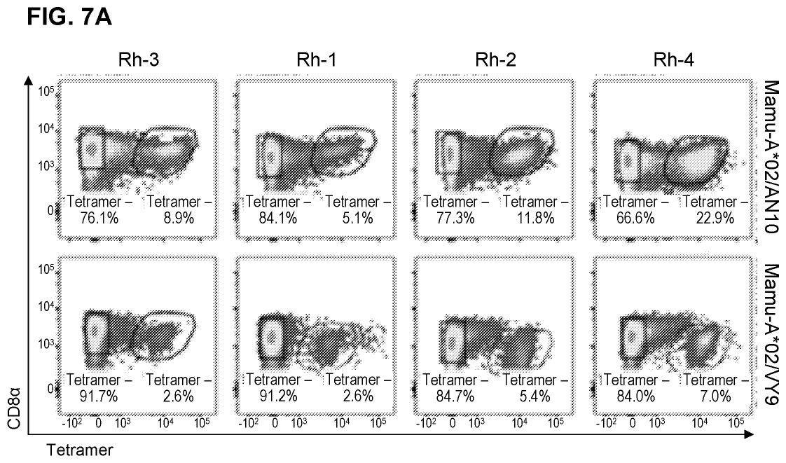

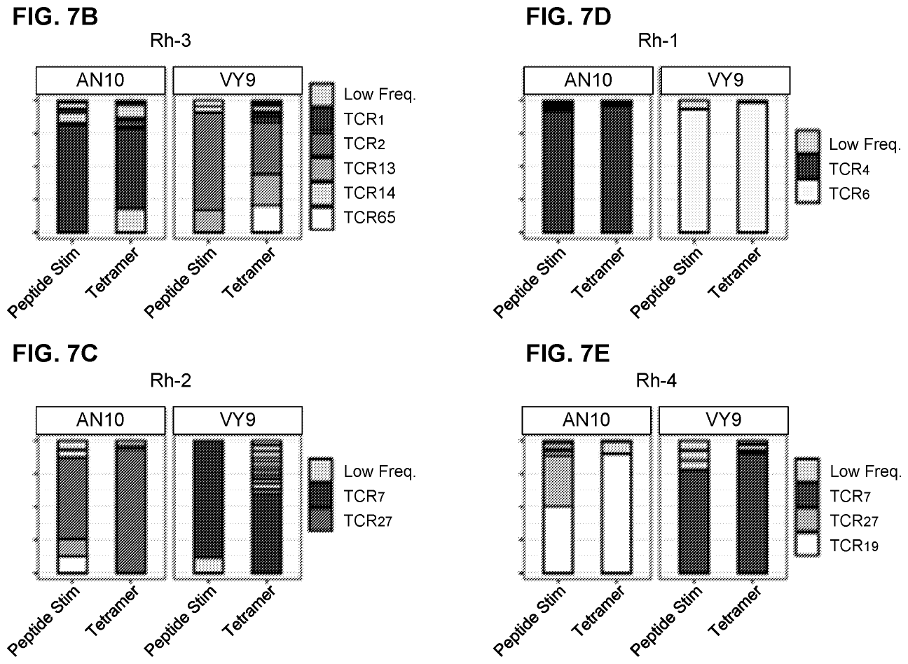

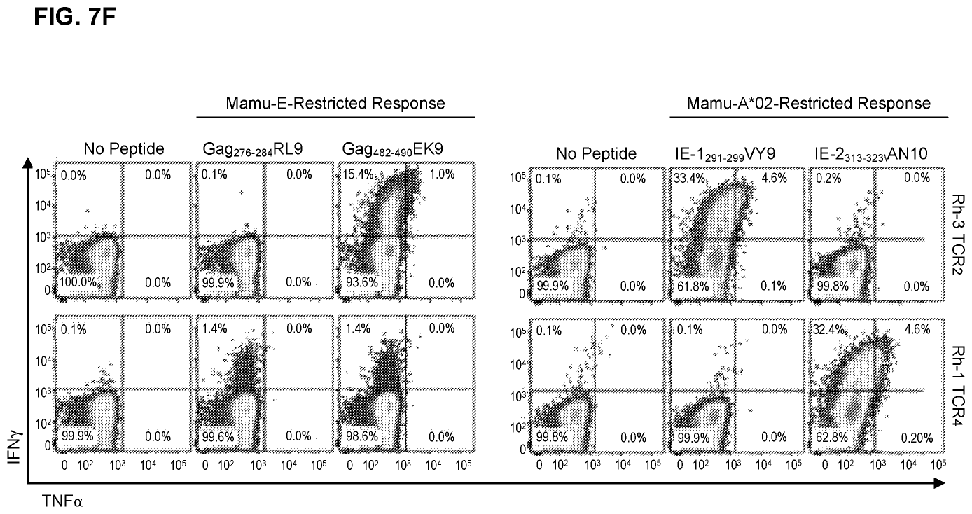

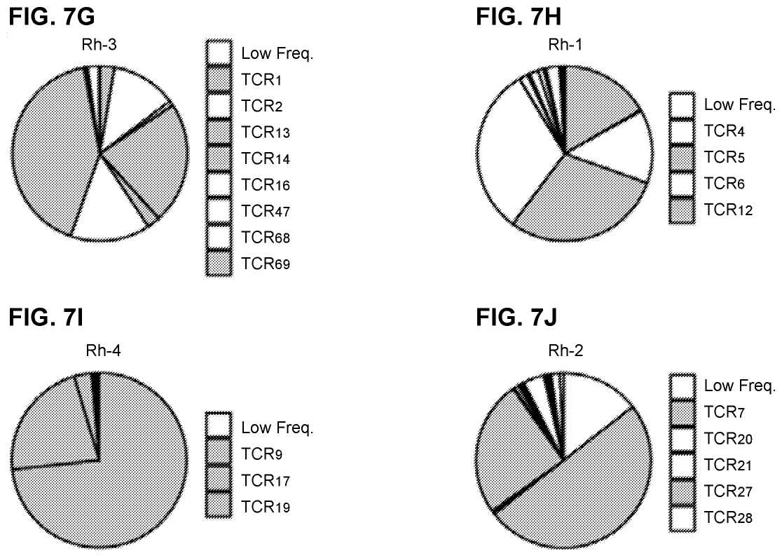

All four of the study RM were naturally RhCMV-infected in the first year of life, and like all RM with natural RhCMV infections would have developed classically MHC-Ia-restricted responses to RhCMV Ags, almost certainly including sizable responses to the RhCMV Immediate Early-1 (IE-1) protein (a highly expressed viral protein that is frequently targeted by T cells). All four study RM expressed the Mamu-A*02 allele, which typically restricts two highly immunodominant IE-1 epitopes: IE₁₃₁₃₋₃₂₃ AN10 and IE₁₂₉₁₋₂₉₉ VY9, and analysis using Mamu-A*02/AN10 and Mamu-A*02/VY9 tetramers revealed that all 4 RM manifested robust CD8+ T cell responses to both epitopes (FIG. 7A).

The CD8+ T cells making up these responses were isolated by sorting on the basis of both Mamu-A*02/AN10/Mamu-A*02/VY9 tetramer binding and sCD69 and stTNF upregulation in response to peptide stimulation by STTS, and sorted cells were analyzed by scRNAseq, as described above. Strikingly and quite surprisingly, some the TCRs identified by this analysis turned out to be the same TCRs previously shown to be triggered by MHC-E-restricted SIVgag supertopes (FIG. 7B-7E). Interestingly, as would be expected with conventional MHC-Ia-restricted CD8+ T cell responses, the TCRs recognizing AN10 and VY9 were distinct, but both also recognized with unconventionally restricted SIVgag supertopes/subtopes.

Functional analysis with TCR transductants confirmed the specific triggering of the relevant TCR by both one of the Mamu-A*02-restricted epitopes and one or both of the MI-IC-E-restricted SIVgag supertopes (FIG. 7F).

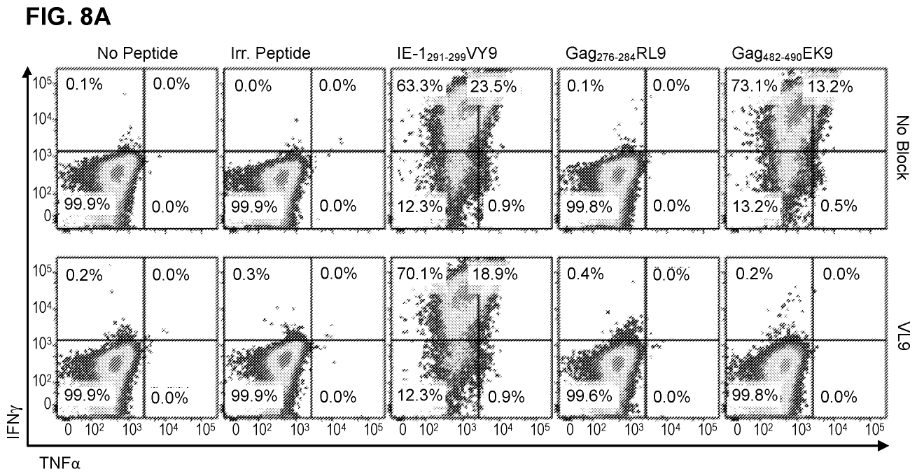

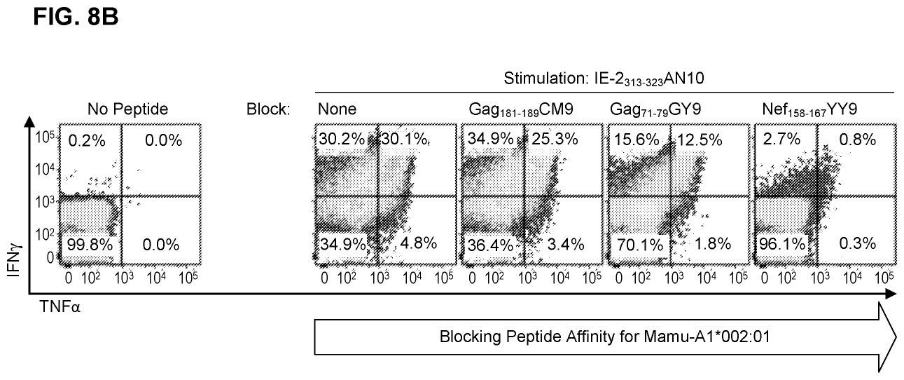

Next, the MHC-Ia restriction was validated by VY9 blocking. Note that VL9 pre-incubation blocks binding and TCR2-mediated recognition of the SIVgag EK9 supertope, but does not block VY9 binding/recognition. The dual reactivities of these transduced CD8+ T cells were confirmed to be distinct in terms of the WIC molecules used for epitope presentation by blocking analysis: recognition of the SIVgag supertopes by TCR transductants were blocked by pre-incubation with the strongly MI-IC-E-binding VL9 peptide, whereas recognition of the Mamu-A*02-restricted IE-1 epitope by the same transductants were not; conversely, TCR transductant recognition of the Mamu-A02 IE-1 epitope could be blocked by Mamu-A*02 binding peptides in proportion to their binding strength (FIGS. 8A-8B). Remarkably, TCRs with both conventional IE-1-specific and unconventional SIVgag-specific reactivity comprised the majority (but not all) of TCRs involved in SIV-infected cell recognition in these four 68-1 RhCMV/SIVgag-vaccinated RM (FIG. 7G-7J).

### Example 8: Dual-MHC-Specificity of CD8+ T Cells can Result from Expression of Two TCR Subunits

As shown in FIGS. 4A-4D, three of the SIVgag supertope-reactive T cell clones identified in the analysis of the 4 study RM expressed two distinct TCR alpha chains and one TCR beta chain, with the potential to form two distinct TCRs. To examine the specificity of each pair, transducants for each pair individually (TCR6-1 and TCR6-2) were generated. Sequences for TCR6-1 and TCR6-2 are shown in Tables 9 and 10.

TABLE 9
TCR6-1 α/β chain sequence.

| Chn | CDR3 |
|---|---|
| α | CAVYGNKLIF (SEQ ID NO: 51) |
| β | CASSYSGINTQYF (SEQ ID NO: 52) |

TABLE 10
TCR6-2 α/β chain sequence.

| Chn | CDR3 |
|---|---|
| α | CGAEIEDGQKLLF (SEQ ID NO: 53) |
| β | CASSYSGINTQYF (SEQ ID NO: 54) |

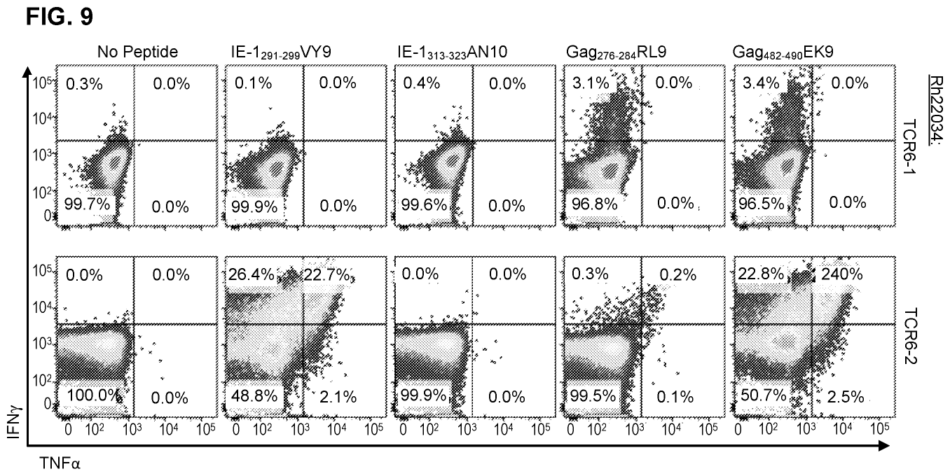

Interestingly, both TCRs recognize Gag₄₈₂₋₄₉₉ EK9 and are broadly (but not identically) cross-reactive with multiple SIVgag subtopes (FIG. 5); however, only one of these pairs (TCR6.2) recognizes an Mamu-A*02 epitope (VY9) (FIG. 9). These data suggest that the conventional VY9 specificity is predominantly TCR alpha chain-dependent, whereas the MHC-E SIVgag specificity is predominantly TCR beta chain-mediated, a unique insight into a possible mechanism by which some of these unusual cross-reactivities might occur.

### Example 9: Functional Avidity of Dual MHC-Specific TCRSs

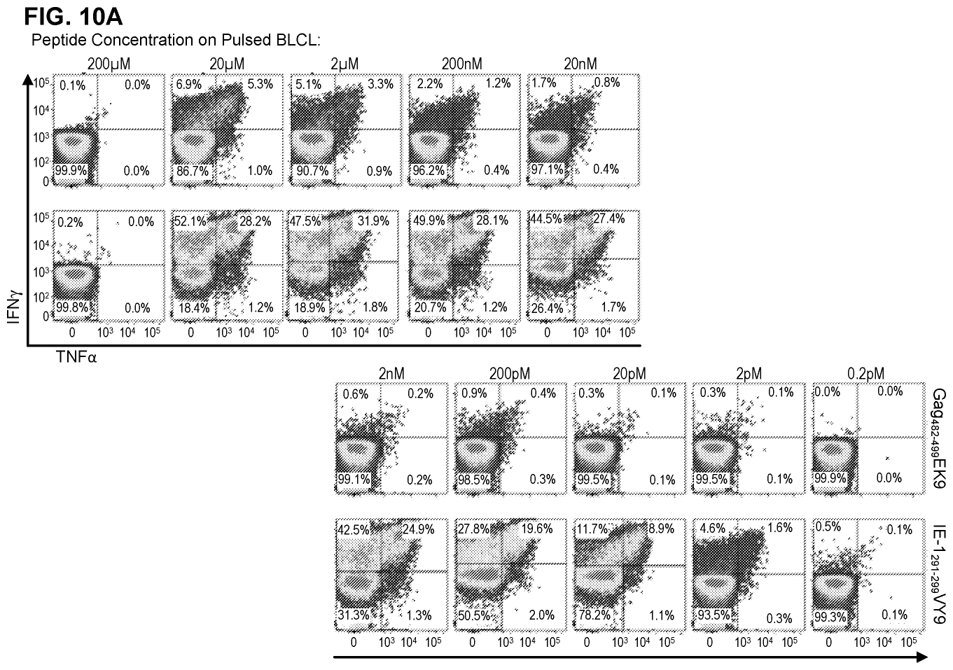

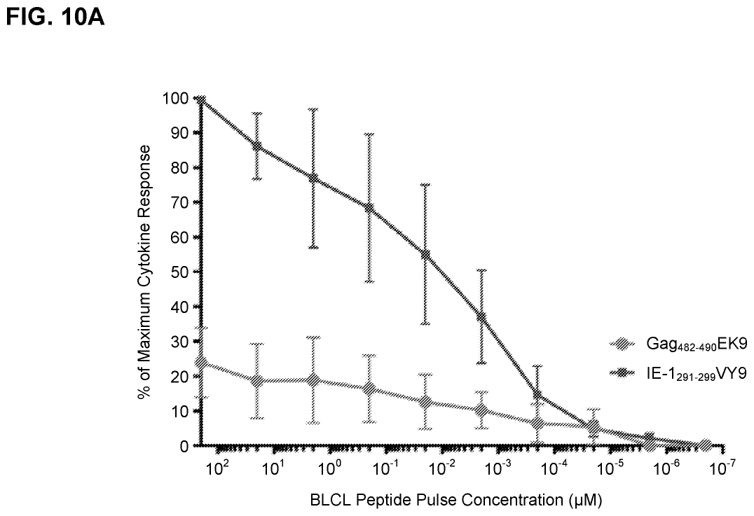

When a given TCR with Mamu-A*02 epitope and MHC-E supertope/subtope multi-specificity are analyzed side-by-side, the response to Mamu-A*02 epitope is often larger (more responding cells) and stronger (more cytokine production) than to the optimal MHC-E supertope responses (see FIGS. 7B-7E, 8A; the TCR6.2 response in FIG. 10, bottom panel being the exception). This is not particularly surprising given that the initial recruitment of these TCR clonotypes in the memory compartment almost certainly occurred during the original wildtype RhCMV infection, but it was important to compare these functional differences in more detail.

As a first step to this objective, side-by-side epitope dilution analysis with TCR2 CD8+ T cell transductants was performed. TCR2 was selected because it has been one of the most consistent and potent TCRs in terms of response to MHC-E supertope and also recognizes the IE-1 VY9 epitope. It was hypothesized that if triggering by the MHC-E supertope was compromised by either weak/unstable binding of the supertope peptide in the MHC-E peptide binding groove, or by low TCR avidity to the supertope-MHC-E complex, one would expect that supertope-mediated triggering would, at high epitope doses, start off similar to triggering by the conventional VY9 epitope (or possibly, less efficient than), but then would fall off fast with epitope dilution, such that demonstrable triggering by the conventional epitope would extend to much lower peptide doses than the unconventional epitope. Intriguingly, this was not what was observed (FIGS. 10A-10B). Even at the highest peptide dose the response to MHC-E supertope triggered fewer TCR2-transduced cells than the conventional VY9 epitope; however, the response of this smaller population fell away at nearly the same rate as the larger population triggered by the conventional epitope. This finding suggests that for this TCR at least, when a transduced cell has the “correct” environment, it can respond to the MHC-E supertope as well as it does to the conventional epitope, but that not all transduced cells have the correct epigenetic landscape to respond. It is important to remember that TCR transduction was performed on peripheral blood CD8+ T cells from control animals, and while the activation required for transduction converts all transductants to a memory phenotype, the origin of these cells is diverse, and thus there is likely heterogeneity in the epigenetic landscape of the transductants. Factors that dictate the ability of the cell to be triggered by MHC-E supertope remain, at this juncture of the project, to be determined. In addition, scRNA was used to determine whether MHC-E supertope non-responding transductants are in fact responding, but with a different activation response that does not include TNF-α or y-IFN production.

### Example 10: Functional Analysis of TCRs Ex Vivo

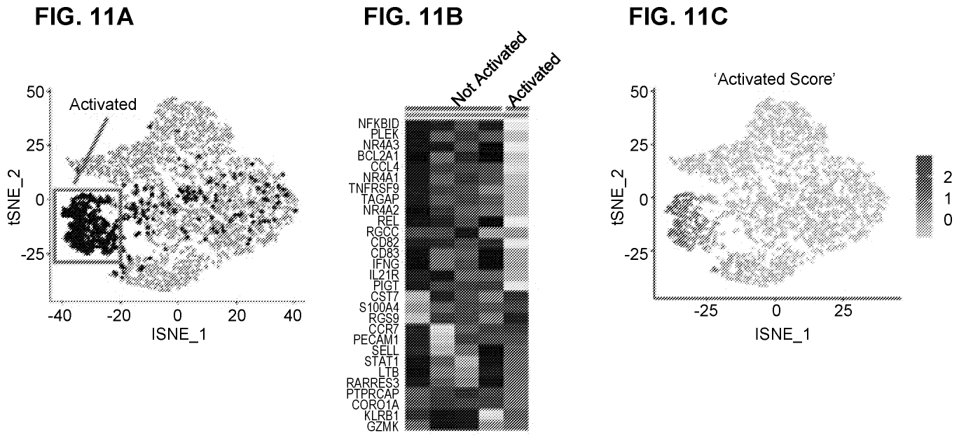

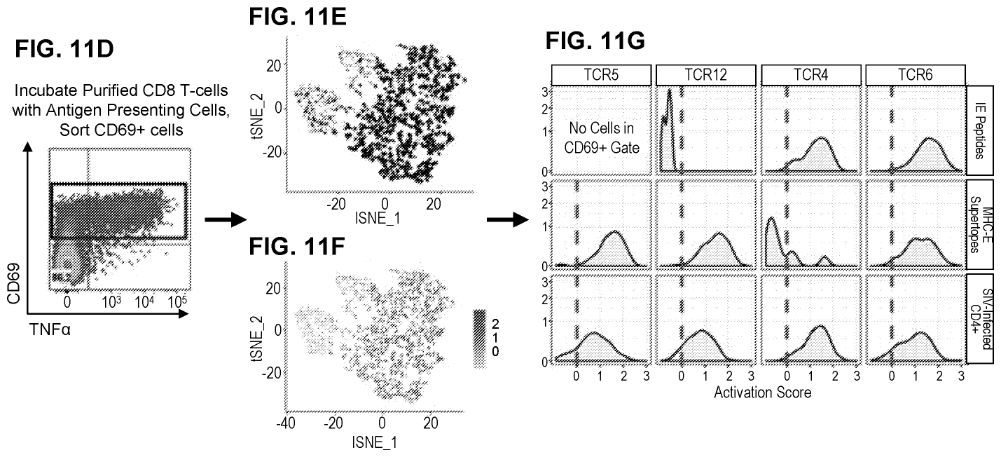

To explore differences in triggering efficiency between conventional and unconventional recognition by cells naturally bearing these TCRs, scRNAseq was used to analyze cells taken ex vivo from the study RM. Although the focus of our use of scRNAseq to this point has been single cell determination of TCR expression, the available data include whole transcriptomes. Ag-activated CD8+ T cells were predominantly sorted prior to scRNAseq analysis (stTNF+/sCD69+), this is primarily done to concentrate the Ag-responsive cells of interest to reduce costs, as the transcriptome of the Ag-responsive cells also provides clear evidence of TCR-mediated activation, easily recognizable by clustering of the responsive cells (with the relevant TCR) in a tSNE plot (FIG. 11A) and by differential gene expression of the presumptively activated “cluster” from the other clusters identified in the tSNE plots, including canonical TCR triggering-induced genes (FIG. 11B). Using the differentially upregulated genes associated with TCR-mediated activation, a composite activation score that provides a quantitative assessment of the cellular response to a given Ag was created (FIG. 11C). Isolated total CD69+ cells were studied, so as to generally enrich activated cells (FIG. 11D-11F, left panels) and then within this mostly activated subset, determine the activation score of CD8+ T cells expressing the relevant TCRs in this RM (See FIGS. 4A-4D; TCR4, 5, 6 and 12) to 3 different stimuli: i) IE-1 peptides VY9+AN10, ii) MHC-E SIVgag supertope peptides RL9+EK9, and iii) SIV-infected cells (FIGS. 11D-11G).

For each assay, total CD69+ cells were sorted (FIG. 11D), which will enrich for activated cells, but contain background and scored cells for activation as above. Again, the cells expressing the cross-reactive TCR cluster with activation (FIGS. 11E-11F). The activation score for the response of CD8+ T cells expressing each indicated TCR to each indicated Ag stimulus was separately evaluated (FIG. 11G). TCR4 and TCR6 have been shown to respond to AN10 or VY9, while TCRS and TCR12 do not. Note that among the two TCRs reacting with MHC-E SIVgag supertopes and MHC-Ia epitopes, the activation distribution suggests more efficient activation (rightward shift) by the MHC-Ia epitopes, which at least for TCR4 is compensated for by the multiple MHC-E SIVgag epitopes presented by the SIV-infected cells.

As noted above (FIG. 9), TCR6+ cells express two TCRs sharing a common beta chain, and thus the response of these cells would likely reflect a composite of both TCRs. As expected from previous analyses (FIGS. 7A-7J), only TCR4 and TCR6 respond to IE-1 VY9+AN10, whereas TCRS and TCR12 are either not in the CD69+ gate at all, or if present, show a sub-zero activation score. In contrast, the TCR6 response to VY9/AN10 is robust and unimodal, despite the fact that only one of this clonotype's two TCRs responds to one of these peptides (VY9). The TCR6 response to the supertope peptides and to SIV-infected cells is slightly, but discernably, left-shifted overall relative the IE-1 VY9 response and appears bimodal suggesting some cells with full activation and others with a lesser induction of the suite of activation genes. The TCR4 response to IE peptide is slightly weaker than the TCR6 response, but the big difference with this TCR is its trimodal responses to MHC-E supertopes, including strong, weak and no response to these peptides, coupled with a robust response to SIV-infected cells. Of note, TCR4 recognizes 4 MHC-E subtopes (FIG. 5; often more strongly than the supertopes) and the SIV-infected cells are likely presenting these additional target epitopes, resulting in a stronger, more uniform response to this almost certainly polyvalent stimulus. Conversely, TCRS is one of the supertope-only TCRs (e.g., no known recognized subtopes: FIG. 5) and the response of this TCR in its native cells to optimal supertope peptides is clearly stronger than to SIV-infected cells. TCR12 has not yet been tested for subtope reactivity, but this TCR shows the same supertope>SIVinfected cell triggering pattern as TCRS, suggesting that it too might be less cross-reactive with subtopes.

Overall, these data suggest that in the context of their native (RhCMV vector-elicited) CD8+ T cells, the MHC-E SIVgag-reactive TCRs are heterogeneous, but clearly can induce a “full” transcriptionally-defined activation response in the majority of cells (similar to that triggered by an MHC-Ia epitope) with either SIVgag supertope peptide or with SIV-infected cells or with both, suggesting that, in contrast to the CD8+ T cell TCR transductants these native (CMV “reared”) CD8+ T cells have the necessary epigenetic landscape to fully respond to these unconventional epitopes.

1. A method of generating CD8+ T cells comprising a multi-specific T cell receptor (TCR), wherein the method comprises:
(a) administering to a subject a recombinant cytomegalovirus (CMV) vector comprising a nucleic acid sequence that encodes a first heterologous antigen, in an amount effective to generate a first set of CD8+ T cells that recognize a first MHC/heterologous antigen-derived peptide complex, wherein the CMV vector does not express an active UL128, UL130, UL146 and UL147 protein or orthologs thereof;
(b) identifying a first CD8+ TCR from the first set of CD8+ T cells, wherein the first CD8+ TCR recognizes the first MHC/heterologous antigen-derived peptide complex;
(c) administering to the subject a second heterologous antigen in an amount effective to generate a second set of CD8+ T cells that recognizes a second MHC/heterologous antigen-derived peptide complex;
(d) isolating one or more CD8+ T cells from the second set of CD8+ T cells;
(e) identifying a second CD8+ TCR from the second set of CD8+ T cells, wherein the second CD8+ TCR recognizes the first MHC/heterologous antigen-derived peptide complex and the second MHC/heterologous antigen-derived peptide complex;
(f) transfecting a third set of CD8+ T cells with an expression vector, wherein the expression vector comprises a nucleic acid sequence encoding a third CD8+ TCR and a promoter operably linked to the nucleic acid sequence encoding the third CD8+ TCR, wherein the third CD8+ TCR comprises CDR3α and CDR3β of the second CD8+ TCR, thereby generating one or more CD8+ T cells that recognize the first MHC/heterologous antigen-derived peptide complex and the second MHC/heterologous antigen-derived peptide complex; and
(g) selecting one or more of the third CD8+ TCRs with the highest avidity for a specific peptide of interest.

2. The method of claim 1, wherein (i) the recombinant CMV vector does not express an active UL18 protein; (ii) the recombinant CMV vector expresses an active UL40protein, or ortholou thereof, and an active US28 protein, or ortholog thereof; or (iii) the recombinant CMV vector does not express an active UL18 protein and expresses an active UL40protein, or ortholog thereof, and an active US28 protein, or ortholog thereof.

3. (canceled)

4. The method of claim 1, wherein the first MHC/heterologous antigen-derived peptide complex is a MHC-II/heterologous antigen-derived peptide complex, a MHC-E/heterologous antigen-derived peptide complex, or a MHC-I/heterologous antigen-derived peptide complex.

5. The method of claim 1, wherein the second MHC/heterologous antigen-derived peptide complex is a MHC-II/heterologous antigen-derived peptide complex or a MHC-E/heterologous antigen-derived peptide complex.

6. The method of claim 1, wherein the subject is a human or non-human primate.

7. The method of claim 1, wherein the recombinant CMV vector is a recombinant human CMV vector or a recombinant rhesus macaque CMV vector.

8. The method of claim 1, wherein the first and/or second heterologous antigen comprises a tumor antigen, pathogen-specific antigen, a tissue specific antigen, or a host-self antigen.

9. The method of claim 8, wherein the tumor antigen is related to a cancer selected from the group consisting of prostate cancer, kidney cancer, lung cancer, pancreatic cancer, mesothelioma, breast cancer, acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, ovarian cancer, colon cancer, renal cell carcinoma, and cervical cancer.

10. The method of claim 8, wherein the pathogen-specific antigen is related to a pathogen selected from the group consisting of human immunodeficiency virus, herpes simplex virus type 1, herpes simplex virus type 2, hepatitis B virus, hepatitis C virus, papillomavirus, Plasmodium parasites, Epstein-barr virus (EBV), Kaposi's sarcoma-associated herpesvirus (KSHV), Human T-lymphotropic virus type 1 (HTLV1), merkel virus (MCV), cytomegalovirus, and Mycobacterium tuberculosis.

11-15. (canceled)

16. The method of claim 1, wherein (i) the first heterologous antigen and second heterologous antigens are the same; or (ii) the first heterologous antigen and second heterologous antigen are different.

17. (canceled)

18. The method of claim 1, wherein the one or more isolated CD8+ T cells from the second set of CD8+ T cells express CD69 and TNFα.

19-87. (canceled)

88. The method of claim 1, wherein the nucleic acid sequence encoding the third CD8+ TCR is identical to the nucleic acid sequence encoding the second CD8+ TCR.

89. The method of claim 1, further comprising isolating one or more CD8+ T cells from a second subject and transfecting the one or more CD8+ T cells with a nucleic acid sequence encoding the selected third CD8+ TCR and a promoter operably linked to the nucleic acid sequence encoding the third CD8+ TCR, thereby generating one or more CD8+ T cells that recognize the first MHC/heterologous antigen-derived peptide complex and the second MHC/heterologous antigen-derived peptide complex.

90-95. (canceled)

96. The method of claim 16, wherein the first subject is a nonhuman primate and the second subject is a human, and wherein (i) the transfected CD8+ T cells comprises a chimeric nonhuman primate-human CD8+ TCR comprising the non-human primate CDR3α and CDR3β of the second CD8+ TCR; (ii) the third CD8+TCR comprises the non-human primate CDR1α, CDR2α, CDR3α, CDR1β, CDR2β, and CDR3β of the second CD8 TCR; (iii) the third CD8+ TCR comprises the CDR1α, CDR2α, CDR3α, CDR1β, CDR2β and CDR3β of the second CD8+ TCR; (iv) the second CD8+ TCR is a chimeric nonhuman primate-human CD8+ TCR comprising the non-human primate CDR3α and CDR3β of the first CD8+ TCR; and/or (v) the third CD8+ TCR is a chimeric CD8+ TCR.

97-100. (canceled)

101. The method of claim 1, wherein administering the recombinant CMV vector to the first subject comprises intravenous, intramuscular, intraperitoneal, or oral administration.

102. A CD8+ T cell comprising the multi-specific TCR generated by the method of claim 1.

103. A method of treating or preventing cancer or treating a pathogenic infection in a subject in need thereof, the method comprising administering the CD8+ T cell of claim 102 to the subject.

104-113. (canceled)

114. The method of claim 1, wherein the MRE contains target sites for microRNAs expressed in endothelial cells.

115. The method of claim 114, wherein the MRE is specific for the miRNA selected from the group consisting of miR126, miR-126-3p, miR-130a, miR-210, miR-221/222, miR-378, miR-296, and miR-328.

116-164. (canceled)

165. The method of claim 1, wherein:
the recombinant CMV vector further comprises a microRNA recognition element (MRE);
the first MHC/heterologous antigen-derived peptide complex is a MHC-E/heterologous antigen-derived peptide complex; and
the second MHC-E/heterologous antigen-derived peptide complex is a MHC-E/heterologous antigen-derived peptide complex.


SEQUENCE LISTING (54 sequences)

SEQ ID NO: 1 - protein, 12 aa, Artificial Sequence
  TCR1-1  TRA CDR3
CAGRDNFNKFYF

SEQ ID NO: 2 - protein, 15 aa, Artificial Sequence
  TCR1-1  TRB CDR3
CASSPREDANYDYTF

SEQ ID NO: 3 - protein, 12 aa, Artificial Sequence
  TCR1-2  TRA CDR3
CARPDSGWQLTF

SEQ ID NO: 4 - protein, 15 aa, Artificial Sequence
  TCR1-2  TRB CDR3
CASSPREDANYDYTF

SEQ ID NO: 5 - protein, 13 aa, Artificial Sequence
  TCR2 TRA CDR3
CAGLGVASNKLTF

SEQ ID NO: 6 - protein, 13 aa, Artificial Sequence
  TCR2 TRB CDR3
CASSYSLKNTQYF

SEQ ID NO: 7 - protein, 13 aa, Artificial Sequence
  TCR13-1 TRA CDR3
CALSNSGYSTLTF

SEQ ID NO: 8 - protein, 12 aa, Artificial Sequence
  TCR13-1 TRB CDR3
CASRKDRSEQYF

SEQ ID NO: 9 - protein, 11 aa, Artificial Sequence
  TCR13-2 TRA CDR3
CASNSGWQLTF

SEQ ID NO: 10 - protein, 12 aa, Artificial Sequence
  TCR13-2 TRB CDR3
CASRKDRSEQYF

SEQ ID NO: 11 - protein, 11 aa, Artificial Sequence
  TCR14  TRA CDR3
CLVRTGNVLTF

SEQ ID NO: 12 - protein, 14 aa, Artificial Sequence
  TCR14  TRB CDR3
CASSPQDRVETQYF

SEQ ID NO: 13 - protein, 14 aa, Artificial Sequence
  TCR16 TRA CDR3
CAVESDTGGFKTVF

SEQ ID NO: 14 - protein, 18 aa, Artificial Sequence
  TCR16  TRB CDR3
CASSQEVGGGGVENTQYF

SEQ ID NO: 15 - protein, 12 aa, Artificial Sequence
  TCR47 TRA CDR3
CMDTGIASKLTF

SEQ ID NO: 16 - protein, 18 aa, Artificial Sequence
  TCR47 TRB CDR3
CASSFYLWGSSGASVLTF

SEQ ID NO: 17 - protein, 13 aa, Artificial Sequence
  TCR68 TRB CDR3
CLVGTGIASKLTF

SEQ ID NO: 18 - protein, 14 aa, Artificial Sequence
  TCR68 TRB CDR3
CASSYRDRAETQYF

SEQ ID NO: 19 - protein, 12 aa, Artificial Sequence
  TCR69 TRA CDR3
CAGRSSYNKLMF

SEQ ID NO: 20 - protein, 15 aa, Artificial Sequence
  TCR69 TRB CDR3
CASSPREDSNYDYTF

SEQ ID NO: 21 - protein, 12 aa, Artificial Sequence
  TCR4  TRA CDR3
CAGRDNFNKFYF

SEQ ID NO: 22 - protein, 15 aa, Artificial Sequence
  TCR4  TRB CDR3
CASSFRDDANYDYTF

SEQ ID NO: 23 - protein, 16 aa, Artificial Sequence
  TCR5  TRA CDR3
CALRDLRNSGNRALVF

SEQ ID NO: 24 - protein, 15 aa, Artificial Sequence
  TCR5  TRB CDR3
CASSPGLGEEETQYF

SEQ ID NO: 25 - protein, 13 aa, Artificial Sequence
  TCR6-1  TRA CDR3
CGAEIEDGQKLLF

SEQ ID NO: 26 - protein, 13 aa, Artificial Sequence
  TCR6-1  TRB CDR3
CASSYSGINTQYF

SEQ ID NO: 27 - protein, 10 aa, Artificial Sequence
  TCR6-2  TRA CDR3
CAVYGNKLIF

SEQ ID NO: 28 - protein, 13 aa, Artificial Sequence
  TCR6-2  TRB CDR3
CASSYSGINTQYF

SEQ ID NO: 29 - protein, 16 aa, Artificial Sequence
  TCR12 TRA CDR3
CALRELLGSGNRALVF

SEQ ID NO: 30 - protein, 14 aa, Artificial Sequence
  TCR12 TRB CDR3
CASSEVGEENTQYF

SEQ ID NO: 31 - protein, 16 aa, Artificial Sequence
  TCR7  TRA CDR3
CAGRAGRGSTLGKLYF

SEQ ID NO: 32 - protein, 18 aa, Artificial Sequence
  TCR7  TRB CDR3
CASSRSEGVTLGADPQYF

SEQ ID NO: 33 - protein, 11 aa, Artificial Sequence
  TCR20 TRA CDR3
CVLIHGNKLIF

SEQ ID NO: 34 - protein, 14 aa, Artificial Sequence
  TCR20 TRB CDR3
CVAGGGGNTAQLFF

SEQ ID NO: 35 - protein, 12 aa, Artificial Sequence
  TCR21 TRA CDR3
CASMDSNYQLIW

SEQ ID NO: 36 - protein, 18 aa, Artificial Sequence
  TCR21 TRB CDR3
CASSQEGIGTGGNAQLFF

SEQ ID NO: 37 - protein, 13 aa, Artificial Sequence
  TCR27 TRA CDR3
CLVGDRRYSTLTF

SEQ ID NO: 38 - protein, 14 aa, Artificial Sequence
  TCR27 TRB CDR3
CASSFRDRAETQYF

SEQ ID NO: 39 - protein, 12 aa, Artificial Sequence
  TCR28 TRA CDR3
CLVRTGGFKTVF

SEQ ID NO: 40 - protein, 14 aa, Artificial Sequence
  TCR28 TRB CDR3
CASSFRDRQETQYF

SEQ ID NO: 41 - protein, 15 aa, Artificial Sequence
  TCR9 TRA CDR3
CIVRRASGGGYVLTF

SEQ ID NO: 42 - protein, 15 aa, Artificial Sequence
  TCR9 TRB CDR3
CASSEGVLAGYDYTF

SEQ ID NO: 43 - protein, 14 aa, Artificial Sequence
  TCR10 TRA CDR3
CAVNAGQAGTALIF

SEQ ID NO: 44 - protein, 16 aa, Artificial Sequence
  TCR10 TRB CDR3
CASSLFFQEGTAQLFF

SEQ ID NO: 45 - protein, 14 aa, Artificial Sequence
  TCR17 TRA CDR3
CALRERFGNEKLTF

SEQ ID NO: 46 - protein, 14 aa, Artificial Sequence
  TCR17 TRB CDR3
CASSLDGGRYDYTF

SEQ ID NO: 47 - protein, 14 aa, Artificial Sequence
  TCR18 TRA CDR3
CALWELGNTGKLIF

SEQ ID NO: 48 - protein, 13 aa, Artificial Sequence
  TCR18 TRB CDR3
CASSLVEGNTQYF

SEQ ID NO: 49 - protein, 13 aa, Artificial Sequence
  TCR19 TRA CDR3
CLLRDSGYSTLTF

SEQ ID NO: 50 - protein, 14 aa, Artificial Sequence
  TCR19 TRB CDR3
CASSYRDRQETQYF

SEQ ID NO: 51 - protein, 10 aa, Artificial Sequence
  TCR6-1 alpha chain CDR3
CAVYGNKLIF

SEQ ID NO: 52 - protein, 13 aa, Artificial Sequence
  TCR6-1 beta chain CDR3
CASSYSGINTQYF

SEQ ID NO: 53 - protein, 13 aa, Artificial Sequence
  TCR6-2 alpha chain CDR3
CGAEIEDGQKLLF

SEQ ID NO: 54 - protein, 13 aa, Artificial Sequence
  TCR6-2 beta chain CDR3
CASSYSGINTQYF